SEQUENCE LISTING (1 sequence)

SEQ ID NO: 1 - protein, 26 aa, Homo sapiens
EQKLISEEDLGSSETVRFQGHHHHHH

(19) United States
(12) Patent Application Publication
(10) Pub. No.: US 2018/0088111 A1
(43) Pub. Date: Mar. 29, 2018

(54) IMMUNOASSAY FOR SOLUBLE PROGRAMMED DEATH-1 (sPD-1) PROTEIN

(71) Applicant: BRISTOL-MYERS SQUIBB COMPANY, Princeton, NJ (US)

(72) Inventors: Yan G. Ni, Yorktown Heights, NY (US); Xiling Yuan, Princeton, NJ (US); Steven P. Piccoli, Princeton, NJ (US)

(21) Appl. No.: 15/565,525

(22) PCT Filed: Apr. 12, 2016
(86) PCT No.: PCT/US2016/027025

§ 371 Date: Oct. 10, 2017

Related U.S. Application Data
(60) Provisional application No. 62/147,211, filed on Apr. 14, 2015.

(51) Int. Cl. G01N 33/566; G01N 33/74

(52) U.S. Cl. CPC G01N 33/566; G01N 33/74

(57) ABSTRACT

This disclosure provides a method for quantifying total soluble Programmed Death-1 (sPD-1) in a sample solution comprising the steps of performing a “sandwich” immunoassay on the sample solution and a series of reference solutions containing known quantities of sPD-1, wherein the immunoassay is performed using (a) a sPD-1 reference antigen in monomeric form; (b) a capture antibody that is capable of binding to sPD-1 in both monomeric and dimeric forms, which binding is essentially unaffected by the presence of the PD-1 ligands, PDL-1, PDL-2, and/or a therapeutic anti-PD-1 antibody; and (c) an electrochemiluminescent-labeled detection antibody that is capable of binding to sPD-1 in both monomeric and dimeric forms, which binding is to a different epitope on sPD-1 than the epitope bound by the capture Ab and is essentially unaffected by the presence of PDL-1, PDL-2 and/or a therapeutic anti-PD-1 Ab.

### CROSS-REFERENCE TO RELATED APPLICATIONS

This application claims priority to U.S. Provisional Application Ser. No. 62/147,211, filed Apr. 14, 2015, the entire content of which is incorporated herein by reference.

Throughout this application, various publications are referenced in parentheses by author name and date. Full citations for these publications may be found at the end of the specification immediately preceding the claims. The disclosures of these publications are hereby incorporated in their entireties by reference into this application in order to more fully describe the state of the art as known to those skilled therein as of the filing date of the application. However, the citation of a reference herein should not be construed as an acknowledgement that such reference is prior art to the disclosed invention.

### FIELD OF THE INVENTION

The invention disclosed herein relates to the development and fit-for-purpose validation of an immunoassay for measuring soluble human PD-1 protein in biological samples, for example, in serum, for supporting clinical studies.

### BACKGROUND OF THE INVENTION

The Programmed Death-1 (PD-1) receptor is expressed on the surface of activated T- and B-lymphocytes as well as thymus resident cells, and is a negative regulator of immune responses (Nishimura et al., 1996). Binding of PD-1 with ligand proteins PDL-1 or PDL-2, which are expressed on tumor and stromal cells, transmits an inhibitory signal that inactivates immune cells (Freeman et al., 2000). Recent progress in delineating mechanism of tumorigenesis supports the hypothesis that tumors can evade normal immune attack through the PD-1 checkpoint pathway via PD-1/PDL-1 interactions (Freeman et al., 2000; Dong et al., 2002). The blockade of checkpoint inhibitors, such as PD-1, on immune effector cells using antagonistic antibodies (Abs) has ushered in an exciting new era in cancer immunotherapy (Pardoll and Drake, 2012; Dublin, 2012; McDermott and Atkins, 2013; Hamid and Carvajal, 2013). Anti-PD-1 Abs, exemplified by nivolumab (OPDIVO®) and pembrolizumab (KEYTRUDA®), have been shown to block the interactions between PD-1 and its ligands, thereby enhancing T-cell responses in vitro (Wang et al., 2014; McDermott and Jimeno, 2015) and eliciting anti-tumor activity in patients with solid tumors (Topalian et al., 2012; Hamid et al., 2013). The availability of clinical assays to quantify soluble factors of the checkpoint pathway in normal and disease sera would enhance understanding of tumorigenesis and facilitate the development of new cancer therapy. Such soluble factors may potentially serve as biomarkers for predicting the efficacy or safety of checkpoint inhibitor drugs, or identifying suitable candidates for treatment with such drugs.

The human PD-1 gene encodes a 288-amino acid (aa) protein (about 55 kDa) comprising a putative signal peptide, an extracellular region with one immunoglobulin like V-type domain, a trans-membrane domain, and a cytoplasmic region (Agata et al., 1996; Shinohara et al., 1994). An alternative splice variant of PD-1 has been reported to produce an isoform that contains only the extracellular domain of the PD-1 protein and exists in sera of normal and rheumatoid arthritis (RA) individuals (Nielsen et al., 2005; Wan et al., 2006). There is some evidence that this soluble form of the PD-1 protein (sPD-1) may serve an autoantibody-like role in circulation that attenuates the negative regulatory effect of PD-1/PDL-1 on T cells (Wan et al., 2006). Interestingly, a recent report showed sPD-1 levels are elevated in early RA patients, and treatment that reduces disease activity concurrently reduces sPD-1 levels (Greisen et al., 2014), raising the possibility of sPD-1 serving as a disease biomarker of RA. Levels of sPD-1 protein in cancer patients have not previously been reported.

In published studies, the sPD-1 protein was measured with a Research Use Only (RUO) commercial kit which has not been validated for clinical use. Thus, there is a need for the development and fit-for-purpose validation (DeSilva et al., 2003; Lee et al., 2006) of a sPD-1 assay to support clinical studies on therapy with anti-PD-1 Abs such as nivolumab. Such an assay should have high analytical sensitivity, a wide dynamic range, low intra- and inter-assay imprecision and low assay bias (percent deviation), and should be capable of quantifying levels of sPD-1 in human sera in the presence of an anti-PD-1 Ab or the PD-1 ligands, PDL-1 and PDL-2.

### SUMMARY OF THE INVENTION

The present disclosure provides a method, specifically an immunoassay, for quantifying total soluble PD-1 (sPD-1) in a sample solution, the method comprising:

(a) immobilizing an anti-sPD-1 capture antibody (Ab) in multiple discrete locations on a solid support, wherein each location contains electrodes designed for applying a voltage waveform effective to trigger electrochemiluminescence (ECL) in a suitable electrochemiluminescent substrate, and further wherein: (i) the capture Ab binds specifically to sPD-1 in both monomeric and dimeric forms; and (ii) binding of the capture Ab to PD-1 is essentially the same in the presence or absence of a therapeutic anti-PD-1 Ab, PDL-1 and/or PDL-2;

(b) contacting the immobilized capture Ab in a sample location with an aliquot of the sample solution and in reference locations with aliquots of a series of reference solutions containing known quantities of sPD-1, under conditions to allow the immobilized capture Ab to bind to sPD-1 in the sample and reference solutions;

(c) contacting each sample and reference location with an electrochemiluminescent-labeled anti-PD-1 detection Ab under conditions to allow the detection Ab to bind to sPD-1 bound by the capture Ab in the sample and reference locations, wherein (i) the detection Ab binds specifically to sPD-1 in both monomeric and dimeric forms; (ii) the detection Ab binds to a different epitope on sPD-1 than the epitope bound by the capture Ab; and (iii) binding of the detection Ab to PD-1 is essentially the same in the presence or absence of a therapeutic anti-PD-1 Ab, PDL-1 and/or PDL-2;

(d) determining the relative quantities of the detection Ab bound to the sample location and the reference locations by measuring ECL; and

(e) quantifying the amount of sPD-1 in the sample solution by comparing the ECL from the detection Ab bound to the sample location relative to the ECL from the detection Ab bound to the reference locations.

In certain preferred embodiments of the immunoassays methods disclosed herein, the solid support is a multiwell plate pre-coated with streptavidin and the capture Ab is a biotin-labeled anti-PD-1 Ab, wherein the capture Ab is immobilized on the plate via a streptavidin-biotin interaction. In further embodiments, the capture Ab is the biotin-labeled monoclonal antibody (mAb) designated MIH4.

In other preferred embodiments, the detection Ab is the electrochemiluminescent-labeled polyclonal Ab designated AF1086.

In yet other preferred embodiments, the immunoassay is performed on the MESO SCALE DISCOVERY® platform.

In certain embodiments, the multiwell plates are pre-coated with the capture Ab and stored at 2-8° C. for a considerable period of, for example, up to about a year before use.

This disclosure also provides a kit for quantifying total soluble sPD-1 in a sample solution, the kit comprising:

(a) an anti-PD-1 capture Ab, wherein (i) the capture Ab binds specifically to sPD-1 in both monomeric and dimeric forms; and (ii) binding of the capture Ab to PD-1 is essentially the same in the presence or absence of a therapeutic anti-PD-1 Ab, PDL-1 and/or PDL-2;

(b) an anti-PD-1 detection Ab, wherein (i) the detection Ab binds specifically to sPD-1 in both monomeric and dimeric forms; (ii) the detection Ab binds to a different epitope on sPD-1 than the epitope bound by the capture Ab; and (iii) binding of the detection Ab to PD-1 is essentially the same in the presence or absence of a therapeutic anti-PD-1 Ab, PDL-1 and/or PDL-2; and

(c) instructions for using the capture Ab and the detection Ab in any one of the immunoassays described herein.

Other features and advantages of the instant invention will be apparent from the following detailed description and examples which should not be construed as limiting. The contents of all cited references, including any scientific articles, newspaper reports, GenBank entries, patents and patent applications cited throughout this application are expressly incorporated herein by reference.

### BRIEF DESCRIPTION OF THE FIGURES

FIG. 1. Characterization of the assay reference standard-recombinant hPD1(25-167)-His protein. A: SDS-PAGE analysis of the purified hPD1(25-167)-His protein expressed in HEK293 cells. Lane 1: molecular weight marker, lane 2: blank, lane 3: 1μg of the purified hPD1(25-167)-His protein. The gel was stained with Simply Blue SafeStain. B: Concentration series of hPDL-1 (0.125-2 μM) binding to captured hPD1(25-167)-His. C: Equilibrium analysis of the binding data yields a K_{D} of 2.8 μM (average value over multiple surfaces).

FIG. 2. sPD-1 MSD assay standard curve. Mean signal units were plotted against sPD-1 protein concentrations with four-parameter logistic regression and 1/Y² weighting. Results are shown as mean and standard error of data collected from 7 validation runs performed in 4 days.

FIG. 3. Detection of endogenous sPD-1 protein and spike recovery in sera from normal and cancer individuals. A: Levels of sPD-1 in serum samples from normal and cancer individuals: normal, n=15; melanoma, n=15; renal cell carcinoma, n=11; squamous lung cancer, n=7; non-squamous lung cancer, n=8; multiple myeloma, n=10; Hodgkin's lymphoma, n=7. B: Spike and recovery study: 500 pg/ml hPD1(25-167)-His protein was spiked into serum samples from normal and cancer individuals: normal, n=10; melanoma n=8; renal cell carcinoma, n=11; squamous lung cancer, n=7; non-squamous lung cancer, n=8; multiple myeloma, n=10; Hodgkin's lymphoma, n=7. % Spike Recovery =100*(sample value post-spike-sample value pre-spike)/spiked value. Results are shown with box & whiskers graphs. The box included data within 5 to 95 percentiles with the median line in the middle and extended values in the whiskers. The dotted lines depict the nominal spiked value±25%.

FIG. 4. Dilution linearity studies in melanoma and spiked normal sera. A: Three melanoma serum samples (M1, M2, M3) were each diluted 1.5-8 fold with assay buffer; recombinant hPD1(25-167)-His protein standard in a SeraSub solution was diluted in same concentration range. B: Three normal human serum samples (N1, N2, N3) were spiked with 5000 pg/ml of human hPD1(25-167)-His and were each diluted 2-256 fold with assay buffer. The back-calculated sPD-1 concentrations (observed concentrations times the dilution factor) were plotted against dilution factors. The dotted lines depict 75-125% range of the nominal spiked value.

FIG. 5. Assay performance and stability of pre-coated and STABILCOAT® treated streptavidin plates. Standard curves (A) and quality control (QC) performance (B) from analytical runs using fresh plates and pre-coated streptavidin plates. The fresh plates were coated with the capture Ab on the day of the assay. The pre-coated plates were coated with capture Ab, treated with different 0.5× or 1× STABILCOAT® and kept at 4° C. or 37° C. for 48 h (no differences observed between the 4° C. and 37° C. groups, and the data were grouped together, n=4). Assay QC performance using 0.5× STABILCOAT® treated plates at a contract research organization (CRO) were also presented (n=18). C: Accelerated stability study of pre-coated streptavidin plates (MSD), showing QC performance in analytical runs using fresh or pre-coated plates (treated with 0.5× STABILCOAT®) stored at 37° C. for 2 weeks. Results are shown as Mean and SD (n=4).

FIG. 6. Assay performance using fresh or frozen standards during validation and clinical studies. A: Standard curves generated by two different analysts using freshly prepared standards (n=2) or pre-prepared standards (n=4) which were kept frozen at −80° C. until use. Results are shown as Mean±standard error of mean. B-D: In-study QC sample performance (using frozen standards). Back-calculated low limit of quantification (LLOQ), low- and mid-level quality control (LQC and MQC) data were collected from assay validation and 4 clinical sample analyses (S1-S4) over a 3 month period. Results are shown as Mean and SD (n ranges 38-76). The dotted lines in the graph depict Mean±2SD values for each QC.

FIG. 7. Effect of PDL-1, PDL-2 and nivolumab on QC sample performance. A: sPD-1 in a melanoma serum sample measured in the absence or presence of 1000 ng/ml (20 nM) of human PDL-1 or PDL-2 protein, respectively. B: QC performance from analytical runs in the absence or presence of 100 μg/ml (685 nM) of nivolumab. Results are shown as mean and SD of each group (n=2-4).

### DETAILED DESCRIPTION OF THE INVENTION

Disclosed herein is the development and validation of an immunoassay for measuring the total soluble forms of PD-1 protein for supporting clinical studies. This total sPD-1 assay quantifies soluble PD-1 in normal and diseased human sera in the presence of the human anti-PD-1 Ab, nivolumab, and the endogenous ligands of PD-1 protein, PDL-1 and PDL-2. The assay validation followed closely the framework for full validation of a biotherapeutic pharmacokinetic biomarker (BM) assay and, therefore, represents a model for analytical validation of other BM assays.

### Terms

In order that the present disclosure may be more readily understood, certain terms are first defined. As used in this application, except as otherwise expressly provided herein, each of the following terms shall have the meaning set forth below. Additional definitions are set forth throughout the application.

An “antibody” (Ab) shall include, without limitation, a glycoprotein immunoglobulin (Ig) which binds specifically to an antigen and comprises at least two heavy chains and two light chains interconnected by disulfide bonds, or an antigen-binding portion thereof. Each heavy chain comprises a heavy chain variable region (VH) and a heavy chain constant region. The heavy chain constant region comprises three constant domains, C_{H1}, C_{H2} and C_{H3.} Each light chain comprises a light chain variable region (V_{L}) and a light chain constant region. The light chain constant region comprises one constant domain, C_{L}, and there are two types of light chain in mammalian Ig's, the kappa and the lambda light chain. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The V_{H} and V_{L} regions contain a binding domain that interacts with an antigen. Within the constant regions of the Ab, comprising two or three heavy chain constant domains in different types of Abs, is the fragment crystallizable region (Fc region) which mediates the binding of the Ig to host tissues or factors, including various cells of the immune system (e.g., effector cells) and the first component (C1q) of the classical complement system, thereby allowing Abs to activate the immune system. Where not expressly stated, and unless the context indicates otherwise, the term “antibody” also includes an antigen-binding portion of any Ig.

The term “monoclonal antibody” (mAb) refers to a non-naturally occurring preparation of Ab molecules of single molecular composition, i.e., Ab molecules whose primary sequences are essentially identical, and which exhibit a single binding specificity and affinity for a particular epitope.

A “polyclonal antibody” is a preparation of Ab molecules that are secreted by different B cell lineages and comprise different Ig molecules that react against a specific antigen, with the Ig molecules collectively recognizing and binding to multiple epitopes of the antigen.

A “capture antibody” refers to an Ab that is attached to a solid support, such as a microwell plate, and used to immobilize on the solid support an antigen specifically recognized by the capture Ab.

A “detection antibody” is an Ab that is used to specifically bind to and thereby detect an antigen that has been immobilized on a solid support, such as a microwell plate. The detection Ab must be capable of being detected, either directly by being linked to a detectable label or indirectly by being specifically bound by a reagent, such as a secondary Ab, that is itself linked to a detectable label.

Abs typically bind specifically to their cognate antigen with high affinity, reflected by a dissociation constant (K_{D}) of about 10⁻⁵ to 10⁻¹¹ M. Any K_{D} greater than about 10⁻⁴ M is generally considered to indicate nonspecific binding. As used herein, an Ab that “binds specifically” to an antigen refers to an Ab that binds to the antigen with high affinity, which means having a K_{D} of about 10⁻⁷ M or lower, preferably about 10⁻⁸ M or lower, more preferably about 5×10⁻⁹ M or lower, about 10⁻⁹ M or lower, or about 5×10⁻¹⁰ M or lower, but does not bind with high affinity to different antigens except where an Ab may be cross-reactive with a common epitope in different antigens. In certain preferred embodiments of the immunoassay disclosed herein, Abs bind to the antigen with a K_{D} between about 5×10⁻⁹ M and about 10⁻¹¹ M.

An “immunoassay” is an Ab-based method for measuring the presence or concentration of an antigen in solution, the method relying on the ability of one or more Abs to specifically recognize and bind to the antigen.

An “electrochemiluminescent” substrate is a substance that generates light when stimulated by electricity in the appropriate chemical environment. An electrochemiluminescent substrate may be used to label a detection Ab, the emission of light upon electrical stimulation allowing highly sensitive detection of the labeled detection Ab.

The “Programmed Death-1” (PD-1) receptor refers to an immunoinhibitory cell surface receptor belonging to the CD28 family. PD-1 is expressed predominantly on previously activated T cells in vivo, and binds to two ligands, PDL-1 and PDL-2. The term “PD-1” as used herein includes human PD-1 (hPD-1) and species homologs of hPD-1. The complete 288-aa sequence of the hPD-1 protein is found under GenBank Accession No. U64863.

“Programmed Death Ligand-1 (PDL-1)” is one of two cell surface glycoprotein ligands for PD-1 (the other being PDL-2) that downregulate T cell activation and cytokine secretion upon binding to PD-1. The term “PDL-1” as used herein includes human PDL-1 (hPDL-1), variants, isoforms, species homologs of hPDL-1, and analogs having at least one common epitope with hPD-L1. The complete aa sequence of hPDL-1 can be found under GenBank Accession No. Q9NZQ7.

“Programmed Death Ligand-2 (PDL-2)” is one of two cell surface glycoprotein ligands for PD-1 (the other being PDL-1) that downregulate T cell activation and cytokine secretion upon binding to PD-1. The term “PDL-2” as used herein includes human PDL-2 (hPDL-2), variants, isoforms, species homologs of hPDL-2, and analogs having at least one common epitope with hPD-L2. The complete aa sequence of hPDL-2 can be found under GenBank Accession No. Q9BQ51.2.

“Soluble Programmed Death-1 (sPD-1)” refers to a protein consisting of essentially the extracellular domain of the PD-1 protein, which exists as a soluble PD-1 isoform in sera. It may be generated, for example, by proteolytic cleavage from membrane-bound PD-1 or as an alternative splice variant of PD-1.

The term “total” sPD-1 refers to the entirety of all forms of sPD-1 in a sample, including, for example, sPD-1 in monomeric or dimeric form, free sPD-1, sPD-1 bound to a therapeutic anti-PD-1 Ab, sPD-1 bound to PDL-1 and/or sPD-1 bound to PDL-2.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject,” “individual” and “patient” are used interchangeably herein.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “about,” “essentially” or “essentially the same” refers to a value, composition or characteristic that is within an acceptable error range for the particular value, composition or characteristic as determined by one of ordinary skill in the art, which will depend in part on how the value, composition or characteristic is measured or determined, i.e., the limitations of the measurement system. For example, “about,” “essentially” or “essentially the same” can mean within 1 or within more than 1 standard deviation per the practice in the art. Alternatively, “about,” “essentially” or “essentially the same” can mean a range of plus or minus 20%, more usually a range of plus or minus 10%. When particular values, compositions or characteristics are provided in the application and claims, unless otherwise stated, the meaning of “about,” “essentially” or “essentially the same” should be assumed to be within an acceptable error range for that particular value, composition or characteristic.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

Immunoassay for Measuring sPD-1 Protein

Enzyme-linked immunosorbent assays (ELISAs) (Voller et al., 1978; Engvall, 1980) are widely used as diagnostic tools in the pharmaceutical industry for the detection and quantification of a specific antigen in a biological sample and for quality control evaluations. Described herein is the development and fit-for-purpose validation of an immunoassay for measuring sPD-1 protein concentration in biological samples to support clinical development of anti-PD-1 therapeutics (Example 3). This immunoassay was developed on the MESO SCALE DISCOVERY® (MSD; Rockville, Md.) platform, which provides an immunoassay system for quantifying analytes using highly sensitive ECL detection instead of an enzyme-mediated detection system used in certain conventional ELISAs.

In the MSD system, high capacity-binding carbon electrodes in the bottom of MULTI-ARRAY® and MULTI-SPOT® microplates allow for easy attachment of biological reagents. These plates are available with different coatings to facilitate the attachment of different types of molecules, e.g., avidin- or streptavidin-coated microplates for attachment of a biotinylated capture reagent, or microplates coated with an anti-species Ab to facilitate the attachment of a capture Ab, are available. MSD assays use electrochemiluminescent labels that are conjugated to detection reagents that bind to the capture Ab-attached analyte in the sample. An electrical voltage is applied to the plate electrodes by an MSD instrument leading to light emission by the labels. Light intensity is then measured, allowing for ultra-sensitive quantification of analytes in the sample.

MSD assays follow a workflow similar to that of a “sandwich” ELISA, comprising the main steps of: coating the plates with a capture reagent such as a capture Ab; blocking the plate to minimize non-specific binding of reagents; adding samples and reference standards or calibrators; adding an electrochemiluminescent-labeled detection reagent such as an electrochemiluminescent-labeled detection Ab; reading the plate; and analyzing the data. This procedure also includes wash steps to remove excess blocking reagents, capture reagent, sample and reference solutions, and detection reagent, as are well known by persons skilled in the art and exemplified in Example 1. Compared to traditional ELISA formats, the MSD platform offers several advantages, including high sensitivity, broad dynamic range, low background and great flexibility as electrochemiluminescent labels are stable, non-radioactive, and can be conveniently conjugated to biological molecules.

As the immunoassay disclosed herein is intended to measure sPD-1 in serum samples, it was important in developing the assay to use as the reference antigen a sPD-1 protein that corresponds to the monomeric form in which PD-1 exists in solution and on the cell surface (see Example 1). Data provided herein suggest that the PD-1 antigen used as the reference in the commercial kit that has been cited in published studies (Wan et al., 2006; Greisen et al., 2014) is a homodimeric form of the extracellular portion of PD-1 that does not reflect the naturally occurring, monomeric form of sPD-1 in serum.

Another critical aspect in developing a sandwich immunoassay was the selection of a “matched pair” of capture and detection Abs that bind to two different, non-overlapping epitopes on the antigen to ensure that neither Ab interferes with the binding of the analyte by the other. Additionally, because the disclosed immunoassay is intended to measure total sPD-1 in the presence of PD-1 ligands and/or a therapeutic anti-PD-1 Ab, it was important to demonstrate that the binding of the capture and detection Abs was not affected by the presence of a therapeutic Ab, PDL-1 or PDL-2. Nine different anti-PD-1 Abs were screened for the appropriate binding properties. Most of these Abs were found to be unsuitable, i.e., they bound weakly or not at all to the monomeric form of sPD-1 (see Example 2). From this screening of PD-1 Abs, the mAb designated MIH4 (available commercially under Catalog No. 17-9969-41 from eBiosciences, San Diego, Calif.) was selected as a capture Ab, and the polyclonal Ab designated AF1086 (available commercially under Catalog No. AF1086 from R&D Systems, Minneapolis, Minn.) was selected as a detection Ab. It was shown that PDL-1 did not interfere with the binding of MIH4 to sPD-1, and various concentrations of capture and detection Abs were evaluated to select optimal concentrations to allow high sensitivity and dynamic range in the presence of nivolumab (Example 2).

Accordingly, the disclosure provides a method for quantifying total sPD-1 in a sample solution comprising the steps of performing a solid phase “sandwich” immunoassay on the sample solution and a series of reference solutions containing known quantities of sPD-1, wherein the immunoassay is performed using (a) a sPD-1 reference antigen in monomeric form, herein exemplified by a recombinant hPD-1(25-167)-His protein; (b) a capture Ab, herein exemplified by the mAb designated MIH4, that is capable of binding to sPD-1 in both monomeric and dimeric forms, which binding is essentially unaffected by the presence of PDL-1, PDL-2 and/or a therapeutic anti-PD-1 Ab; and (c) an electrochemiluminescent-labeled detection Ab, herein exemplified by the ruthenium complex-labeled polyclonal Ab designated AF1086, that is capable of binding to sPD-1 in both monomeric and dimeric forms, which binding is to a different epitope on sPD-1 than the epitope bound by the capture Ab and is essentially unaffected by the presence of PDL-1, PDL-2 and/or a therapeutic anti-PD-1 Ab. One of skill in the art would recognize that the immunoassay disclosed herein is not limited to the particular sPD-1 antigen and the particular matched pair of capture and detection Abs disclosed herein, or to being performed on the MSD platform.

The assay sensitivity (LLOQ) of the disclosed immunoassay was 100 pg/ml and the dynamic range was 100-10,000 pg/ml. Evaluations of a large number of serum samples from ongoing clinical studies and a commercial source showed that the assay sensitivity and dynamic range were sufficient for sPD-1 measurement in normal and cancer subjects. The intra- and inter-assay imprecision, defined by variance derived from back-calculated concentrations of LQC, MQC and HQC, was <15%. The accuracy of the assay, defined by total error of LQC, MQC and HQC, was <20%. The intra- and inter-assay imprecision of assay LLOQ of 100 pg/ml was ≦25%.

A LLOQ sample, made of a normal human serum pool with a low level of sPD1, was included in all validation and sample analysis runs since the baseline sPD-1 concentration in normal healthy individuals, as determined using this assay, was close to the assay LLOQ. Both LQC and MQC samples were prepared from pooled human sera which mimic the actual sample matrix and serve as good monitors of assay variability. The use of frozen standards and pre-coated assay plates kept at 4° C. did not affect assay performance, nor did the use of different analysts, different plate washers or plate readers—a testimony to the robustness of the assay. This assay measures total sPD-1 in human serum since nivolumab and the endogenous PD-1 ligands, PDL-1 and PDL-2, do not interfere with the assay.

Reagent Characterization

Careful selection and characterization of key assay reagents is critical to ensure immunoassay performance (Haulenbeek and Piccoli, 2014). During assay development, two candidate reference standard proteins were evaluated: hPD1(25-167)-His, which contains the extracellular portion (aa's 25-167) of the full length PD-1 receptor, and hPD1 (25-167)-3S-IG, a PD-1-Fc chimeric protein that is similar to the commercial reagent used in published studies (Wan et al., 2006; Greisen et al., 2014) (see Example 1). The goal was (1) to gain information regarding protein identity, glycosylation content and purity of the reference protein, and (2) to have a reference standard which closely mimics the endogenous analyte in structure and function. Size exclusion chromatography-multiangle light scattering (SEC-MALS) analysis showed that hPD1(25-167)-His is a glycosylated monomeric protein in solution phase. Further, its binding affinity to sPDL-1 is similar to published results (cf. Lee, 2009). The same SEC-MALS study showed that hPD1 (25-167)-3S-IG formed a homodimer in solution. Since it has been documented that PD-1 is monomeric in solution and on the cell surface (Cheng et al., 2013), the hPD1(25-167)-His was selected as reference standard for the assay (see Example 1).

A pair of Abs was selected as suitable for the assay, and reagent characterization efforts also included Ab binding affinity assessments when it was possible (see Example 2). The binding affinity of the coating Ab MIH4 to sPD-1 was determined to be 2.8 nM by surface plasmon resonance (SPR). The binding affinity of AF1086 to sPD-1 was not examined since it was a polyclonal Ab. In addition, each lot of the biotinylated coating Ab and ruthenium-labeled detection Ab was examined for its purity and its labeling reagent molar incorporation ratio using size exclusion chromatography (SEC) and Matrix-Assisted Laser Desorption Ionization Time-of-Flight (MALDITOF) technology (data not shown). Together, these detailed characterization efforts enabled a better understanding of assay performance and ensured consistent, reproducible assay results over time and across different reagent lots.

Matrix Effect

As is commonly the case in many high sensitivity biomarker assays, it proved challenging to find a serum matrix having no detectable sPD-1 protein. Therefore, recombinant human sPD-1 proteins were reconstituted in a serum substitute, SERASUB®, a buffered synthetic polymer solution which is similar to serum and plasma with respect to specific gravity, viscosity and osmolality. To determine whether there is similar proportionality in immunoreactivity between the recombinant sPD-1 protein standard in SERASUB® and the endogenous sPD-1 protein in serum matrix (“parallelism”), a dilution linearity study was conducted using samples with high levels of endogenous sPD-1 proteins. Results from this study showed that the dilution curve of endogenous sPD-1 in sample matrix was parallel to that of the reference standard in SERASUB® solution in the same concentration range (Example 3). These findings support the view that recombinant sPD-1 standards in buffer offer a valid proportional scale for the relative quantitative measurement of the endogenous sPD-1 protein in serum.

Soluble factors in the sample matrix may cause assay interference due to nonspecific binding (matrix effect). This was investigated by spike and recovery studies on 15 lots of normal human serum and about 10 serum lots each of 6 cancer types. No significant differences in spike recovery were observed in disease sera versus that in normal sera using this assay (Example 3).

Embodiments of the sPD-1 Immunoassay

Although the immunoassay described herein was developed using MSD equipment and reagents (Example 3), it is not limited to being performed on the MSD platform. Accordingly, this disclosure provides an immunoassay for quantifying total sPD-1 in a sample solution comprising:

(a) immobilizing an anti-PD-1 capture Ab in multiple discrete locations on a solid support, wherein each location contains electrodes designed for applying a voltage waveform effective to trigger electrochemiluminescence (ECL) in a suitable electrochemiluminescent substrate, and further wherein:
(i) the capture Ab binds specifically to sPD-1 in both monomeric and dimeric forms; and
(ii) binding of the capture Ab to PD-1 is essentially the same in the presence or absence of a therapeutic anti-PD-1 Ab, PDL-1 and/or PDL-2;

(b) contacting the immobilized capture Ab in a sample location with an aliquot of the sample solution and in reference locations with aliquots of a series of reference solutions containing known quantities of sPD-1 in monomeric form, under conditions to allow the immobilized capture Ab to bind to sPD-1 in the sample and reference solutions;

(c) contacting each sample and reference location with an electrochemiluminescent-labeled anti-PD-1 detection Ab under conditions to allow the detection Ab to bind to sPD-1 bound by the capture Ab in the sample and reference locations, wherein
(i) the detection Ab binds specifically to sPD-1 in both monomeric and dimeric forms;
(ii) the detection Ab binds to a different epitope on sPD-1 than the epitope bound by the capture Ab; and
(iii) binding of the detection antibody to PD-1 is essentially the same in the presence or absence of a therapeutic anti-PD-1 Ab, PDL-1 and/or PDL-2;

(d) determining the relative quantities of the detection Ab bound to the sample location and the reference locations by measuring ECL; and

(e) quantifying the amount of sPD-1 in the sample solution by comparing the ECL from the detection Ab bound to the sample location relative to the ECL from the detection Ab bound to the reference locations.

In certain embodiments of this immunoassay, the solid support is a multiwell plate. In certain preferred embodiments, the multiwell plate is a MSD MULTI-ARRAY® or MULTI-SPOT® plate. MULTI-ARRAY® microplates comprise multiple wells, each containing a carbon electrode integrated in the bottom of the well for applying an electrical voltage to measure ECL from an electroluminescent label. MULTI-SPOT® plates offer up to 10 spots within each well for increased throughput and assay multiplexing. These microplates combine ECL and arrays to bring speed and a high density of information to immunoassays.

Immobilization of the capture Ab on the solid support can be accomplished by direct, passive adsorption. However, passive adsorption may be disadvantageous, causing problems including improper orientation of the bound Ab, denaturation and poor immobilization efficiency. Accordingly, in preferred embodiments, in order to increase the sensitivity and specificity of the assay, the capture Ab is bound to the solid support via a specific interaction between the support and the Ab. In certain embodiments, the capture antibody is bound to the solid support via an antibody-antibody interaction. For example, MSD supplies MULTI-ARRAY® plates pre-coated with an anti-species Ab (e.g., an Ab that recognizes the Fc region of rabbit Abs) that binds specifically to any capture Ab generated in the host species against which the anti-species Ab was raised (e.g., any capture Ab raised in a rabbit and, therefore, comprising a rabbit Fc region). In other embodiments, the capture Ab is immobilized on the solid support via an interaction between the Fc region of the Ab and Protein A and/or Protein G attached to the solid support, or an interaction between Protein L on the solid support and the variable region of a kappa light chain of the Ab. In further embodiments, the capture Ab is immobilized on the solid support via an interaction between biotin and a biotin-binding protein such as streptavidin, NeutrAvidin or avidin, i.e., a streptavidin-biotin interaction, a NeutrAvidin-biotin interaction, or an avidin-biotin interaction. Immobilization employing such specific interactions generally orients the capture Ab properly to preserve its antigen binding capability.

Accordingly, in certain embodiments, the solid support is pre-coated with avidin, streptavidin, NeutrAvidin, Protein A and/or Protein G, Protein L, or an anti-species Ab that binds specifically to the capture Ab. Several parameters play a role in determining the best plate type for an assay. For example, the avidin-coated plates supplied by MSD have a high binding capacity due to their hydrophilic surface but tend to offer lower signals and sensitivity. These plates are ideal for assays that require a large dynamic range. Alternatively, streptavidin-coated plates have relatively lower binding capacity but frequently exhibit lower non-specific binding, especially with complex sample matrices, and provide higher assay signals and sensitivity. Small spot streptavidin plates provide the highest assay signals and, therefore, superior sensitivity. In certain embodiments, the MSD MULTI-ARRAY® plate or MULTI-SPOT® plate used in any of the disclosed immunoassays is pre-coated with avidin, streptavidin or an anti-species Ab that binds to the constant region of the capture Ab. In preferred embodiments, a MSD streptavidin-coated MULTI-ARRAY® or MULTI-SPOT® plate is used.

In certain embodiments of the immunoassay disclosed herein, immobilizing the capture Ab comprises:

(a) incubating a streptavidin-coated multiwell plate with a protein blocking reagent; and

(b) incubating the blocked plate with a biotin-labeled anti-PD-1 capture Ab under conditions to allow the Ab to be immobilized on the plate via a streptavidin-biotin interaction.

In certain preferred embodiments, the protein blocking reagent is BLOCKER™ Casein.

Either monoclonal or polyclonal Abs can be used as the capture and detection Abs in sandwich immunoassay systems. MAbs have the advantage that they can be produced in essentially infinite amounts. Additionally, their inherent monospecificity toward a single epitope permits fine detection and quantification of small differences in antigen. A polyclonal Ab is often used as the capture Ab to pull down as much of the antigen as possible, followed by the use of a mAb as the detection Ab to provide improved specificity. In practice, the type of Ab selected may be determined by which individual Abs meet the requirements for a matched pair of capture and detection Abs, e.g., in the present immunoassay, the ability to bind to the monomeric form of sPD-1, the ability to bind to different epitopes on the antigen, and the insensitivity of sPD-1 binding to the presence of PDL-1, PDL-2 and therapeutic PD-1 Abs.

In certain embodiments, the capture Ab is a polyclonal Ab. In other embodiments, the capture Ab is a mAb. In further embodiments, the monoclonal capture Ab binds to human sPD-1 with a K_{D} of about 10⁻⁸ M or lower. Preferably, the monoclonal capture Ab binds to human sPD-1 with a K_{D} of about 3×10⁻⁹ M or less, 2.8×10⁻⁹ M or less, about 10⁻⁸ to about 10⁻¹⁰ M, or about 3×10⁻⁹ M to about 10^{×11} M. In other preferred embodiments, the capture Ab and the detection Ab each bind to monomeric and dimeric forms of sPD-1 with essentially the same affinity. In preferred embodiments, the monoclonal capture Ab binds to human sPD-1 in the presence of a therapeutic anti-PD-1 Ab which is nivolumab or pembrolizumab. In other preferred embodiments, the capture Ab is the mAb designated MIH4 (available commercially under Catalog No. 17-9969-41 from eBiosciences).

In certain aspects of the disclosed invention, conditions to allow the immobilization of the capture Ab to the solid support comprise incubation at room temperature, typically about 20-25° C., for about 2 h with shaking. However, good results may be obtained with a variety of incubation periods and temperatures. Thus, in certain embodiments, the capture Ab is added to the solid support and incubated at room temperature or at 37° C. for at least about 15 min, at least about 30 min, at least about 1 h, at least about 2 h, or about 1 to about 2 h. In other embodiments, incubation is at about 2 to about 8° C. overnight, i.e., for at least about 12, at least about 15, at least about 18 h, or for about 12 to about 18 h.

In certain embodiments, the reference solutions comprise known quantities of sPD-1 protein in monomeric form dissolved in a synthetic serum substitute. In certain preferred embodiments, the monomeric form of sPD-1 protein is a recombinant hPD-1 (25-167)-His protein. In other embodiments, the synthetic serum substitute comprises a buffered protein-free polymer solution that is equivalent or similar to human serum with respect to specific gravity, viscosity and osmolality. In certain preferred embodiments, the synthetic serum substitute is SERASUB®.

In certain embodiments, prior to addition to the locations on the solid support, the sample solution and reference solutions are diluted with a diluent comprising a protein-containing buffered solution, preferably an immunoglobulin-containing solution, that blocks non-specific binding of the detection Ab to the sample and reference locations. In certain preferred embodiments using the MSD platform, the diluent is MSD Diluent 2 and the sample solution and reference solutions are diluted about 4-fold with MSD Diluent 2.

In certain embodiments, conditions to allow binding of the immobilized capture Ab to sPD-1 in the sample and reference solutions comprises incubation at 37° C., or preferably room temperature (about 20-25° C.), for about 2 h with shaking. In other embodiments, the sample and reference solutions are added to the solid support and incubated at room temperature or at 37° C. for at least about 15 min, at least about 30 min, at least about 1 h, at least about 2 h, or about 1 to about 2 h. In other embodiments, incubation is at about 2 to about 8° C. overnight, i.e., for at least about 12, at least about 15, at least about 18 h, or for about 12 to about 18h.

In certain aspects of this invention, the detection antibody is a mAb. In other aspects, it is a polyclonal Ab. In certain preferred embodiments, the detection Ab is the polyclonal Ab designated AF1086 (available commercially under Catalog No. AF1086 from R&D Systems). In certain other embodiments, conditions to allow the binding of the detection Ab to sPD-1 comprises incubation at 37° C., or preferably room temperature (about 20-25° C.), for about 1 h with shaking. Incubation times of at least about 15 min, at least about 30 min, at least about 0.5 to about 1 h, at least about 2 h, or about 1 to about 2 h may also be used. Alternatively, incubation is at about 2 to about 8° C. overnight, i.e., for at least about 12, at least about 15, at least about 18 h, or for about 12 to about 18 h.

ECL enables highly sensitive and selective analytical assays due to several advantageous features, including the absence of a background optical signal, precise control of reaction kinetics offered by controlling the applied potential, compatibility with solution-phase and thin-film formats, and opportunities to enhance intensity with nanomaterials such as metallic nanoparticles and nanotubes. Accordingly, ECL-generating substrates have been used extensively as labels on biological molecules in bioassays. In the production of ECL, electrochemically generated intermediates undergo a highly exergonic reaction to give rise to an electronically excited state that emits light (Forster et al., 2009). ECL used in immunoassays is commonly produced by reacting electrogenerated tris-2,2′-bipyridylruthenium(III) [Ru(bpy)₃]³⁺ (where bpy refers to 2,2′-bipyridine) with tripropylamine (TPA) to create an excited state that emits light at about 610 nm. The use of osmium polypyridine complexes is also being investigated in ECL-based assay development. Osmium systems are more photostable than their ruthenium analogs, they usually oxidize at less anodic potentials, and their longer emission wavelength may be more suitable for some applications (e.g., there may be less spectral overlap with the absorption spectrum of whole blood). In addition, certain osmium complexes, e.g., [Os(phen)₂(dppene)]²⁺ (phen=1,10-phenanthroline and dppene=bis(diphenylphosphino)ethene), exhibit a more intense ECL emission than does [Ru(bpy)₃]²⁺ (Forster et al., 2009).

In certain embodiments, the detection Ab used in the disclosed immunoassay is labeled with an electrochemiluminescent label which is a ruthenium complex-containing label. In certain preferred embodiments using the MSD platform, the electrochemi-luminescent label is a ruthenium complex-based SULFO-TAG™ label. In other embodiments, the electrochemiluminescent label is an osmium complex-based label. In certain embodiments, determining the quantity of the electrochemiluminescent-labeled anti-PD-1 detection antibody comprises adding to each location on the solid support an aliquot of a medium suitable for triggering ECL. In further embodiments, the medium is a TPA-containing buffer solution. In certain preferred embodiments using the MSD format, the TPA-containing buffer solution is MSD Read Buffer Solution.

ECL may be measured using a variety of image sensors, including a photodiode array, charge coupled device (CCD), complementary metal-oxide-semiconductor (CMOS), back-side illuminated CMOS (BSI-CMOS), or N-type metal-oxide-semiconductor (NMOS) image sensor. In certain embodiments of the disclosed immunoassay, the ECL is measured using a CCD image sensor. In further embodiments, the CCD image sensor comprises an ultra-low noise CCD camera and a telecentric lens. In certain preferred embodiments using the MSD platform, the ECL is measured using a MSD SECTOR® Imager. In other preferred embodiments, the MSD SECTOR® Imager is a MSD SECTOR® 6000 Imager.

In certain embodiments, the sample solution is a biological sample from a subject. In further embodiments, the biological sample is blood, plasma, serum or urine. In certain preferred embodiments, the subject is a human. In other preferred embodiments, the biological sample is human serum.

In certain preferred aspects, the disclosed immunoassay is performed using the MESO SCALE DISCOVERY® (MSD) platform. Accordingly, the present disclosure provides an immunoassay for quantifying total sPD-1 in a sample solution comprising:

(a) incubating a streptavidin-coated MULTI-ARRAY® or MULTI-SPOT® plate with a BLOCKER™ Casein reagent;

(b) incubating the blocked plate with a capture antibody which is biotin-labeled anti-PD-1 mAb MIH4 under conditions to allow the Ab to be immobilized on the plate via a streptavidin-biotin interaction;

(c) diluting aliquots of the sample solution and aliquots of reference solutions 4-fold in MSD Diluent 2 and adding to separate arrays or spots on the plate one or more aliquots of the sample solution (sample arrays or spots) and aliquots of a series of reference solutions (reference arrays or spots) under conditions to allow the immobilized capture Ab to bind to sPD-1 in the sample and reference solutions, wherein the reference solutions comprise known quantities of a monomeric sPD-1 protein dissolved in SERASUB®;

(d) adding to each sample and reference array or spot a detection Ab which is SULFO-TAG™-labeled anti-PD-1 polyclonal Ab AF1086 under conditions to allow the detection Ab to bind to sPD-1 bound by the capture Ab in the sample and reference solutions;

(e) adding to each array or spot an aliquot of MSD Read Buffer Solution and measuring the ECL from each array or spot to determine the relative quantities of the SULFO-TAG™-labeled detection Ab bound to each reference and sample array or spot; and

(f) quantifying the amount of sPD-1 in the sample solution by comparing the ECL from the detection Ab bound to the sample array or spot relative to the ECL from the detection Ab bound to the reference arrays or spots.

A person skilled in the art would immediately recognize that certain reagents and equipment used in this immunoassay can be substituted by similar or equivalent non-MSD reagents and equipment with no significant deleterious effects on the sensitivity, dynamic range, precision and general utility of the assay.

Use of Bulk-Prepared Assay Reagents

Biomarker sample analysis supporting late phase clinical trials often involves processing a large number of samples collected over many years and at different analytical labs. Consistency in assay performance over time and across different labs is challenging, and very important, for delivering high quality, informative clinical data. Accordingly, bulk-prepared streptavidin plates (MSD) pre-coated with capture Ab and frozen standards and QC samples were used in a variant of the disclosed immunoassay (see Example 4). This allows the use of the same batches of standards, QCs and assay plates to ensure consistent assay performance over time and across different laboratories. In addition, these measures also increased assay efficiency by significantly reducing assay duration from about 8-9 h to about 6 h.

It was found that treating the capture Ab-pre-coated microplates with an immunoassay stabilizer solution such as STABILCOAT® is critical for maintaining the integrity of these plates. Accelerated stability studies showed that the pre-coated plates after STABILCOAT® treatment can be kept at 37° C. for up to two weeks. Based on previous studies, these plates are likely to generate consistent sPD-1 measurements for up to 1 year if kept at 4° C. (Weiss et al., 2009). The long-term stability of these plates has been conducted in real time, and thus far, their stability for at least 4 months at 4° C. has been confirmed.

Accordingly, in certain embodiments of the immunoassay disclosed herein, preparation of the solid support, e.g., a multiwell plate, containing the immobilized capture Ab is completed at least about 12 h prior to use, i.e., prior to contacting the immobilized capture antibody with the sample and reference solutions. In other embodiments, immobilization of the capture Ab on the solid support is completed about 12 h to at least 2 years prior to use. In other embodiments, this immobilization is completed at least about 1 day, at least about 1 year, at least about 2 years, about 12 h to about 2 years, about 1 day to about 1 year, or about 1 day to about 6 months prior to use.

In certain embodiments comprising pre-coating the solid support with the capture Ab, this pre-coating comprises:

(a) immobilizing the anti-PD-1 capture Ab in multiple locations on the solid support;

(b) adding to the solid support an immunoassay stabilizer solution and incubating for at least about 15 min at room temperature (typically about 20-25° C.); and

(c) drying the solid support at room temperature for at least about 1 h. It should be understood that each of the multiple locations on the solid support to which the capture Ab is immobilized in step (a) contains an electrode designed for applying a voltage waveform effective to trigger ECL in a suitable electrochemiluminescent substrate, wherein (i) the capture Ab is capable of binding specifically to sPD-1 in both monomeric and dimeric forms; and (ii) binding of the capture Ab to PD-1 is essentially the same in the presence or absence of a therapeutic anti-PD-1 antibody, PDL-1 and/or PDL-2. It should also be understood that in step (b) the stabilizer solution is added to the solid support to allow interaction with the entire Ab-coated surface. In certain preferred embodiments, the immunoassay stabilizer solution is STABILCOAT®.

The incubation period can also vary. Thus, in certain embodiments, after addition of the immunoassay stabilizer solution the solid support is incubated at about 20-25° C. for about 15 min to about 2 h, or for at least about 1 h. In certain alternative embodiments, the pre-coated solid support in step (c) is dried by (1) placing it in a humidity controlled chamber (less than about 15% humidity) until dry (about 4 to about 24 h; (2) placing it at about 30 to about 40° C. in a vacuum oven for about 1 to about 4 h; or (3) air-drying it at room temperature (about 20-25° C.) overnight. The solid support, pre-coated with the capture Ab, may be stored at room temperature, but is preferably stored at about 2 to about 8° C., until use. The various embodiments described herein for the immunoassay using freshly prepared, capture Ab-coated solid supports, e.g., the use of streptavidin-coated multiwell plates with a biotin-labeled capture Ab, matched MIH4 and AF1086 capture and detection Abs, ruthenium-based electrochemiluminescent-labeled detection Ab, and the MSD platform are applicable to the immunoassay employing bulk-prepared assay reagents and capture Ab-pre-coated solid supports.

This disclosure also provides a semi-automated adaptation of the immunoassay described herein, wherein steps for measuring aliquots of reagents and adding reagents to the sample and reference locations on the solid support are performed on a robotic sample processor. In certain embodiments, the robotic sample processor is a Tecan Freedom Evo® processor (Tecan, Research Triangle Park, N.C.).

The methods described herein, including among other features, ECL-based detection of the sPD-1 analyte, and the use of bulk-prepared frozen reference solutions and capture Ab-pre-coated microplates, is a highly sensitivity immunoassay having a broad dynamic range, low intra- and inter-assay imprecision, and high accuracy. In certain embodiments of this assay, the sensitivity, as measured by the back-calculated low limit of quantification (LLOQ), is about 500 pg/ml or lower. In preferred embodiments, the assay sensitivity (LLOQ) is about 100 pg/ml or lower. In further embodiments, the dynamic range is about 100 to about 10,000 pg/ml. In certain other embodiments, the intra- and inter-assay imprecision as measured by variance derived from back-calculated concentrations of low-, mid-, and high-level quality control (LQC, MQC and HQC) is less than about 15%. In further embodiments, the intra- and inter-assay imprecision of assay LLOQ of 100 pg/ml is about 25% or less. In yet further embodiments, the assay accuracy as measured by total error of LQC, MQC and HQC is less than about 20%.

Comparison with Existing sPD-1 Immunoassay and Clinical Application

Soluble PD-1 protein levels have been reported to be elevated in RA and are associated with disease activity (Wan et al., 2006; Greisen et al., 2014). In addition, a study that prospectively examined the relationship with plasma sPD-1 levels and hepatitis B virus load and the development of hepatocellular carcinoma was recently published (Cheng et al., 2014). In these published studies, baseline sPD-1 levels in healthy control human sera were reported to be in the low ng/ml range, which is higher than the level observed (about 200 pg/ml) using the assay disclosed herein. This difference may be, at least in part, due to the differences in assay methodologies. In the published studies, levels of sPD-1 were measured with a Research Use Only (RUO) commercial kit using a purified recombinant hPD-1-Fc chimeric protein as reference standard (Wan et al., 2006; Greisen et al., 2014). Based on the data disclosed herein on hPD1(25-167)-3S-IG (see Example 1), the hPD-1-Fc chimera is likely to be a homodimer. In contrast, the hPD1(25-167)-His protein used as reference standard in the present assay is a monomeric protein and a better mimic of the endogenous sPD-1 protein in human circulation (Zhang et al., 2004). The commercial RUO assay and the present assay also used different capture Abs. Although the two assays shared the same detection Ab, this Ab was labeled with biotin in the commercial assay whereas it was labeled with a ruthenium-based electrochemi-luminescent label in the present assay. The well known advantages of ECL, specifically relating to its high sensitivity, broad dynamic range and low backgrounds are important advantages of the immunoassay disclosed herein. In addition, the two assays also used different buffers. The RUO kit is likely to have matrix interference issues if it were used in clinical studies, since dilution linearity could not be established in several human serum samples using this kit (data not shown).

The present study provides the first report of sPD-1 levels in human cancer. Clinical studies on samples from multiple clinical trials have shown that standard curve range and QC levels were appropriate for samples from multiple cancer types both pre-and post-nivolumab treatment (specific results not shown).

### Kits

Also within the scope of the present invention are kits comprising a matched pair of a capture Ab and a detection Ab for use in an immunoassay for quantifying total sPD-1 in a biological sample. Kits typically include a label indicating the intended use of the contents of the kit and instructions for use. The term label in this context includes any writing, or recorded material supplied on or with the kit, or which otherwise accompanies the kit. Accordingly, this disclosure provides a kit for quantifying total sPD-1 in a sample solution, the kit comprising:

(a) an anti-PD-1 capture Ab, wherein
(i) the capture antibody binds specifically to sPD-1 in both monomeric and dimeric forms; and
(ii) binding of the capture Ab to PD-1 is essentially the same in the presence or absence of a therapeutic anti-PD-1 Ab, PDL-1 and/or PDL-2;

(b) an anti-PD-1 detection Ab, wherein
(i) the detection Ab binds specifically to sPD-1 in both monomeric and dimeric forms;
(ii) the detection Ab binds to a different epitope on sPD-1 than the epitope bound by the capture Ab; and
(iii) binding of the detection Ab to PD-1 is essentially the same in the presence or absence of a therapeutic anti-PD-1 Ab, PDL-1 and/or PDL-2; and

(c) instructions for using the capture Ab and the detection Ab in the immunoassay disclosed herein.

The disclosure also provides different embodiments of this kit corresponding to all the various embodiments of the matched pair of capture and detection Abs described herein. For example, in certain embodiments of the kit, the capture Ab is labeled with biotin for use in immobilizing the Ab to a solid support to which a biotin-binding protein is attached. In other embodiments, the capture Ab is pre-coated on a multiwell plate and stored for an extended period up to about a year before use. In certain preferred embodiments, the capture Ab is MIH4 (eBiosciences, San Diego, Calif.; Catalog No. 17-9969-41). In further embodiments, the detection Ab is labeled with an electrochemi-luminescent label, preferably a ruthenium complex-based label. In certain preferred embodiments, the detection Ab is AF1086 (R&D Systems, Minneapolis, Minn.; Catalog No. AF1086). In yet other embodiments, the kit further comprises a device for collection of a biological sample.

The present invention is illustrated by the following examples which should not be construed as further limiting.

### EXAMPLE 1

### Expression, Purification and Characterization of Soluble Extracellular PD-1 Domain Proteins

### Materials and Methods

Human Serum Specimens

Sera from normal healthy individuals or patients with different cancer types (melanoma, renal cell carcinoma, squamous and non-squamous lung cancer, Hodgkin's lymphoma, multiple myeloma) were purchased from Bioreclamation (Westbury, N.Y.).

Construction of Expression Vectors for Soluble PD-1 Extracellular Domain Proteins

A cDNA clone encoding human PD-1 pre-protein residues 1-167 (NCBI mRNA RefSeq NM_005018) linked at the C-terminus with a Myc-TVMV-His fusion (-EQKLISEEDLGSSETVRFQGHHHHHH) was generated by PCR as a SalI-BamHI fragment and cloned into a GATEWAY® pENTR™ vector (Invitrogen, Carlsbad, Calif.). The protein derived from this construct [hPD1(25-167)-Myc-TVMV-His] was designated hPD1(25-167)-His, reflecting the amino acid sequence of the extracellular domain of the mature PD-1 protein.

In a similar fashion, a clone was constructed encoding the same region of human PD-1 fused at its C-terminus to a portion of human IgG1 Fc region (UniProt P01857) beginning from the hinge (with 3 Cys→Ser substitutions) and including the C_{II2} and C_{II3} domains. The protein derived from this construct was designated hPD1(25-167)-3S-IG.

Both of these DNA sequences were introduced into a pTT22gate-based vector by GATEWAY® LR recombination.

HEK293 Expression of Soluble PD-1 Extracellular Domain Proteins

HEK293 cells (HEK293-6E) at 1×10⁶ cells/ml were transfected with the hPD1(25-167)-His or hPD1(25-167)-3S-IG GATEWAY® destination vectors using a Durocher expression system with a 1:2 DNA:PEI (polyethylenimine) ratio. The transiently-transfected cells were cultured in F17 expression medium, and the conditioned media were harvested by sedimentation 5 days post-transfection and filtered through 0.45 μm filters. For hPD1(25-167)-His, the cell density at harvest was about 4×10⁶ cells/ml, and the cell viability was >95%. For hPD1(25-167)-3S-IG, the cell density at harvest was about 3×10⁶ cells/ml, and the cell viability was >90%.

Purification of hPD1(25-167)-His Protein

The conditioned medium was concentrated and buffer exchanged by tangential flow filtration into phosphate-buffered saline (PBS) with 200 mM additional NaCl and 20 mM imidazole (pH 8) using a Pellicon-2 TFF system and Ultracel-5 membrane (Millipore, Billerica, Mass.). This sample was loaded onto 2×5 ml HisTrap HP columns (GE Healthcare, Princeton, N.J.) connected in series, washed with the same buffer, and eluted with a steep linear gradient to 300 mM imidazole in PBS with 200 mM additional NaCl (pH 8). The protein eluted with high imidazole was pooled, concentrated by centrifugal ultrafiltration to about 8 mg/ml, and loaded onto a 26 mm×600 mm Superdex 200 preparative SEC column (GE Healthcare) equilibrated with PBS (pH 7.5). Based on absorbance at 280 nm, there was a small amount of large molecular sized material that eluted early, and this portion was discarded. The majority of protein eluted from the SEC column in a peak centered at 225 ml. The hPD1(25-167)-His protein fractions from this peak were pooled, divided into aliquots at 1.5 mg/ml, flash frozen in liquid nitrogen, and stored at −80° C.

Purification of hPD1(25-167)-3S-IG Protein

The conditioned medium was concentrated and buffer exchanged by tangential flow filtration into PBS (pH 7.4) using a Pellicon-2 TFF system and Ultracel-10 membrane (Millipore). This sample was loaded onto 2×5 ml HisTrap rProteinA columns (GE Healthcare) connected in series, and the columns were washed with PBS (pH 7.4). The hPD1(25-167)-3S-IG protein was eluted with 80 mM sodium acetate (pH 3.0). The pH of the solution containing the eluted protein was immediately adjusted to about pH 7.5-8.0 by the drop-wise addition of about ⅛th volume of 1 M Tris-HCl (pH 8.0). The protein was concentrated to about 17 mg/ml by centrifugal ultrafiltration and loaded onto a 16 mm×600 mm Superdex 200 preparative SEC column (GE Healthcare) equilibrated with PBS (pH 7.4). As observed by absorbance at 280 nm, the protein eluted from the column in a peak centered on 74 ml with essentially no large aggregates in earlier eluting fractions. The pool of peak fractions containing purified hPD1(25-167)-3S-IG protein at 1.9 mg/ml in PBS pH 7.4 was divided into aliquots, flash frozen in liquid nitrogen, and stored at −80° C.

SDS-PAGE Analysis of Purified hPD1(25-167)-His Protein

Purified hPD1(25-167)-His protein was diluted with 4× LDS sample buffer (Invitrogen) and 1 M dithiothreitol (DTT) to a final concentration of 1× LDS/100 mM DTT. The sample was heated to 65° C. for 30 min, cooled on ice, and then loaded onto a NuPAGE 4-12% acrylamide Bis-Tris gel (Invitrogen) with Novex Sharp pre-stained molecular weight standards (Invitrogen). The gel was electrophoresed at about 200 V for 35 min and then removed and stained with Simply Blue SafeStain (Invitrogen) according to the manufacturer's instructions.

SEC VIALS Analysis of Purified hPD1(25-167)-His Protein

The molecular weight (MW) and estimate of the carbohydrate content of the purified PD-1 extracellular domain proteins were determined by analytical SEC coupled with ultraviolet (UV) absorption, refractive-index, and multi-angle light scattering (MALS) detection. Isocratic separations were performed on a Shodex Protein KW 803 column (Shodex, New York, N.Y.) connected to a UFLC system (Shimadzu, Kyoto, Japan) consisting of a degasser, isocratic pump, chilled sample holder with injector, UV/visible detector, and column oven, in a buffer containing 200 mM K₂HPO₄, 150 mM NaCl (pH 6.8), with 0.02% sodium azide running at 0.5 ml/min. Data were obtained from three online detectors connected in series: the Shimadzu SPD-20 dual wavelength UV/visible spectrophotometer monitoring 280 nm, followed by a mini-Dawn TREOS three angle laser light scattering detector and an Optilab TrEX interferometric refractometer (Wyatt Technologies, Santa Barbara, Calif.). Data were collected and analyzed using ASTRA 6 (Wyatt Technologies) and Lab Solutions (Shimadzu) software.

The carbohydrate content was determined by standard procedures in ASTRA 6 based upon the contributions of protein and carbohydrate to the absorbance at 280 nm, and the refractive index signal observed for the glycoprotein as it was eluted from the SEC column.

High Resolution Mass Spectrometry (HRMS)

Purified hPD1(25-167)-His protein (8 μg) was incubated with 4 μl of protein deglycosylation mix (Cat. No. V4931, Promega, Wis.), prepared according to the manufacturer's specifications, at 37° C. for 16 h with constant shaking. 15.0 μl of the resultant solution was mixed with 10.0 μl of 10 mM tri(2-carboxyethyl)-phosphine (TCEP). The mixture was then injected (5 μl) into an Acquity H-class Bio ultra high pressure liquid chromatography system (UPLC; Waters Co., Milford, Mass.) fitted with a Kinetex C8 column (2.1×50 mm, 1.7 μm; Phenomenex Inc., Torrance, Calif.). The mobile phases of 0.1% formic acid aqueous (A) and 0.1% formic acid in acetonitrile (B) were delivered under a gradient program: 20% B to 80% B over 7.0 min (curve factor 4), 80% B to 90% B over 0.5 min (curve factor 4), followed by re-equilibration. The flow rate was set to 0.3 ml/min and the column was held at 80° C. The column eluent was introduced into a Bruker Daltonik MaXis 4G q-TOF mass spectrometer (Bruker Daltonik GmbH, Bremen, Germany). The ionization source was set to positive polarity mode with a capillary voltage at 4.5 kV, nebulizer pressure at 1.6 bar, dry gas flow at 9.01/min, and temperature at 220° C. The mass analyzer was calibrated between 300 and 2900 m/z and spectra were collected at 1.0 Hz. Profile mass spectra were summed from 3.4 to 3.7 min, smoothed, baseline subtracted and deconvoluted using a maximum entropy equation in Compass DataAnalysis 4.2 (Bruker Daltonik).

Matrix-Assisted Laser Desorption Ionization Time-of-Flight Mass Spectrometry (MALDITOF-MS)

A saturated solution of 2,5-dihydroxy acetophenone (2,5-DHA) (Sigma-Aldrich, St. Louis, Mo.) was made by adding the matrix to 1.0 ml of an acetonitrile:water:trifluoroacetic acid (80:20:0.1) solution. The solution was sonicated and deemed saturated when solids still remained after 5 min of sonication. Purified hPD1(25-167)-His protein (1.0 μl) was spotted onto a stainless steel 384MTP target plate (Bruker Daltonik). The saturated 2,5-DHA solution (1.0 μl) was added to the sample spot on the target plate and mixed by pipetting the solution up and down for 3 cycles. The resultant spot was allowed to dry before analysis. Samples were then analyzed on a Bruker AutoFlex III MALDI-TOFMS (Bruker Daltonik) equipped with a 355 nm YAG SMARTBEAM® laser. Spectra were collected in linear positive ion mode.

Calibration Standards and Quality Control Samples Preparations

The hPD1(25-167)-His protein stock in PBS-10% glycerol was first diluted with 1× Reagent Diluent (R&D Systems, Minneapolis, Minn.) and then with SERASUB® (CST Technologies, NY) to prepare sPD-1 standards at concentrations of 100, 200, 400, 1000, 2,000, 4,000 and 10,000 pg/ml. These standards were stored in aliquots at −80° C. until use.

Low-, mid- and high-level quality control (LQC, MQC and HQC) samples were prepared by spiking sPD-1 [hPD1(25-167)-His] reference standards into a normal human serum pool (prescreened for low sPD-1 levels) to prepare QCs of 500, 1,500 and 5,000 pg/ml respectively. In addition, a low limit of quantification (LLOQ) QC sample was generated using a normal human serum pool with the targeted endogenous sPD-1 concentration of about 100 pg/ml. All QC aliquots were kept frozen at −80° C. until use.

Binding of Human PDL-1 to hPD1(25-167)-His Protein

Surface plasmon resonance (SPR) experiments to characterize the binding of hPDL-1 to hPD1(25-167)-His were performed using a PROTEON™ XPR36 instrument (BioRad, Hercules, Calif.). The surface was activated by injecting nickel (II) ions at 30 μl/min for 120 s. All analyses were performed at 25° C. in a PBS-0.05% TWEEN® solution.

To determine binding affinity of hPDL-1 to soluble PD-1, the hPD1(25-167)-His protein was captured via its His-tag on a PROTEON™ HTG chip surface (Bio-Rad). Different concentrations (6.25, 12.5, 25, 50, 100 μg/ml) of hPD1(25-167)-His were injected at 30 μl/min for 300 s to generate 5 different surface densities. The ligand capture response levels ranged from 480 to 1200 RU. The analyte hPDL-1 protein (0.125-2 μM) was injected over the surface at 30 μl/min for 180 s. The surface was regenerated with 300 mM EDTA (pH 8.5) following BioRad's recommendations. The binding data were analyzed using the PROTEON MANAGER™ Software from Bio-Rad.

### Results

Generation and Characterization of the sPD-1 Reference Standards

To generate human sPD-1 reference material, two different recombinant human sPD-1 fusion proteins, hPD1(25-167)-His and hPD1(25-167)-3S-IG, that contain the extracellular domains of PD-1 (aa's 25-167) and a hexa-His or a human IgGl Fc domain at the C-terminus, respectively, were expressed. The hPD1(25-167)-3S-IG protein is similar to the commercially available PD-1 Fc chimeric protein which has been used in published studies (Wan et al., 2006; Greisen et al., 2014). Both recombinant sPD-1 proteins were expressed in transiently-transfected HEK293 cells and purified.

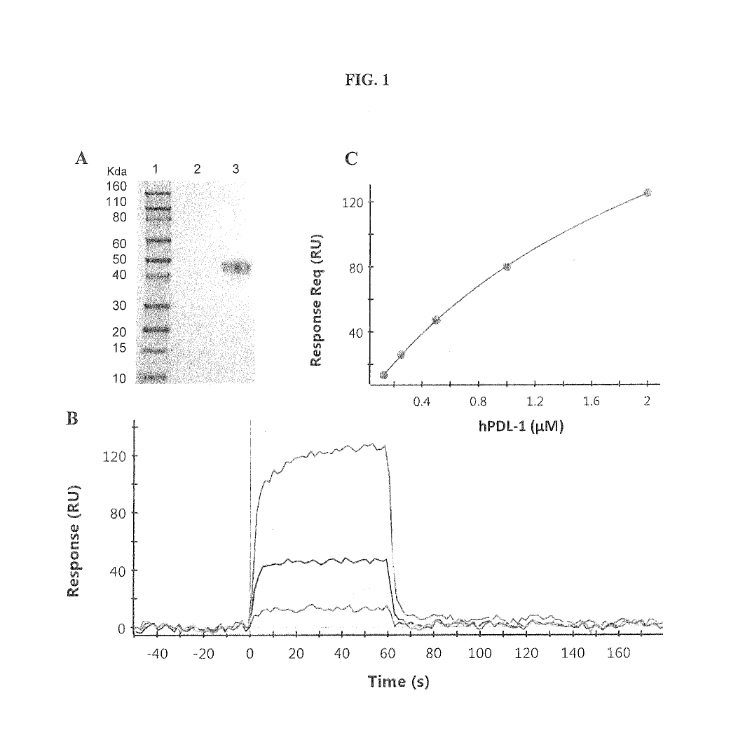

The MW and carbohydrate content of the purified hPD1[25-167]-His and hPD-1-3S-IG proteins were estimated by SEC-MALS. This analysis indicated that both proteins were glycosylated, consistent with the presence of four predicted N-linked glycosylation sites in the human PD-1 extracellular domain and their expression in HEK293 cells. SEC-MALS analysis also indicated that hPD1(25-167)-His and hPD1 (25-167)-3S-IG in PBS solution were glycosylated monomeric and dimeric proteins, respectively (see Table 1). Since structural and biophysical studies have shown that PD-1 is monomeric both in solution as well as on cell surface (Zhang et al., 2004), the monomeric hPD1(25-167)-His protein was selected as the reference standard for the sPD-1 assay to closely mimic the endogenous protein. SDS-PAGE analysis showed that the purified hPD1(25-167)-His protein is of a single form with >95% purity and an apparent MW of about 45 kDa (FIG. 1A).

TABLE 1
Characterization of hPD1(25-167)-His and hPD1(25-167)-3S-IG

| | hPD1(25-167)-His | hPD1(25-167)-3S-IG |
|---|---|---|
| Predicted Monomer MW | 19.0 kDa | 42.6 kDa |
| Predicted Dimer MW | 38.0 kDa | 85.3 kDa |
| Observed Glyco- | 33.1 kDa | 114.0 kDa |
| conjugate MW | | |
| Calculated Protein MW | 20.0 kDa | 93.0 kDa |
| Mass as Carbohydrate | 39.6% | 18.4% |
| by SEC-MALS | | |
| Mass Fraction Recovered | 90.3% | 99.9% |
| by SEC-MALS | | |
| Glyco-conjugate MW by | 30.9 kDa | |
| MALDI-TOF | | |
| Protein MW by HRMS | 19.0 kDa | |
| Mass as Carbohydrate by | 38.5% | |
| MALLDI-TOF and HRMS | | |

High resolution mass spectrometry (HRMS) was used to obtain a more accurate determination of the mass of the hPD1(25-167)-His protein. The protein was subjected to deglycosylation using PNGase F (Promega) and injected onto a liquid chromatography-mass spectrometry (LC-MS) system equipped with an electrospray ionization (ESI) source and quadrupole time-of-flight (q-TOF) mass analyzer. The experimentally observed mass of 19009.8620 Da represents a mass accuracy of <3ppm as compared to the calculated average mass [M +H] ion for the amino acid sequence. The average mass value of the glycosylated hPD1(25-167)-His protein was determined to be 30.9 kDa using matrix-assisted laser desorption ionization time-of-flight mass spectrometry (MALDITOF-MS). Therefore, the carbohydrate content was calculated to be 38.5% by mass.

The functional binding affinity of hPD1(25-167)-His protein to human PDL-1 was assessed using SPR. In this study, hPD1(25-167)-His protein was captured on a HTG chip surface by its His-tag. Soluble, human PDL-1 was injected into the mobile phase as an analyte. The K_{D}, determined to be 2.8 μM (FIGS. 1B and C), was similar to the previously reported affinity of PDL-1 to PD-1 protein (Cheng et al., 2013).

### EXAMPLE 2

### Selection of Antibody Pair and Concentrations

### Materials and Methods

Antibodies and Recombinant Proteins

Anti-PD-1 Abs were purchased from commercial sources or produced internally (see Table 2 for details on Ab name, source, isotype and antigen information). Recombinant human PDL-1 and PDL-2 proteins were purchased from R&D Systems.

Binding of Anti-PD-1 Antibody MIH4 to hPD1(25-167)-His Protein

SPR experiments were performed to characterize the binding of hPD1(25-167)-His to MIH4, a mouse mAb against human PD-1. The experiments were performed at 25° C. using a Biacore T200 instrument (GE Healthcare). All the consumables were obtained from GE Healthcare. Running buffer for all steps was HBS-EP (0.01 M HEPES pH 7.4, 0.15 M NaCl, 3 mM EDTA, 0.005% v/v Surfactant P20). The hPD1(25-167)-His protein was coupled to a Series S Sensor Chip CMS utilizing 1-ethyl-3-(3-dimethylaminopropyl) carbodiimide hydrochloride (EDC) direct coupling. Target ligand densities were 300 to 400 RU for kinetic measurements.

For each flow cell, different concentrations (0.195-50 nM) of MIH4 were injected at 30 μl/min for 240 s to generate 8 different surface plasmon sonograms for kinetic calculations. The flow cells were regenerated with glycine (pH 2.0) between each injection of anti-PD-1. The response levels obtained for the analyte ranged from 0.2 to 49 RU. The data were analyzed with the Biacore T200 Evaluation software (GE Healthcare). Kinetic fit data with χ² values less than 0.1 were accepted.

### Results

The ability of several anti-human PD-1 Abs, produced either internally or from commercial sources, to bind to both monomeric (hPD1(25-167)-His) and dimeric (hPD1 (25-167)-3S-IG) forms of human sPD-1 proteins in the presence of nivolumab was tested by standard ELISA. The results are shown in the last two columns of Table 2. Two Abs, MIH4 and AF1086, that bound to both monomeric and dimeric forms of sPD-1 in the presence of nivolumab, were selected as capture and detection Abs, respectively.

The binding affinity of MIH4 to sPD-1 protein was 2.8 nM as determined by SPR analysis (Table 3). The effect of PDL-1 on the binding of hPD1(25-167)-His to MIH4 was also studied using SPR. When the hPD1(25-167)-His protein was immobilized on the chip, its binding affinity to MIH4 Ab in the presence of 2M of PDL-1 protein was found to be 2.9 nM, indicating that the presence of PDL-1 did not affect hPD1(25-167)-His and MIH4 binding.

TABLE 2
Antibodies evaluated during development of the human sPD-1 MSD assay.

| | | | | Binding to | Binding to |
|---|---|---|---|---|---|
| | | | | hPD1(25- | hPD1(25- |
| Antibody | Source | Isotype | Antigen | 167)-His | 167)-3S-IG |
| 26D5 | Internal | Mouse IgG1 | hPD-1 Fc dimer | Weakly | Yes |
| Ab58812 | Abcam | Rabbit polyclonal | 16-aa peptide from | No | No |
| | | IgG | near the centre of | | |
| | | | hPD-1 | | |
| Ab52587 | Abcam | Mouse IgG1, κ | Human T/NK cell | Weakly | Weakly |
| | | | leukemia | | |
| Ab89828 | Abcam | Mouse IgG1, κ | Full length hPD-1 | Weakly | Weakly |
| J105 | eBioscience | Mouse IgG1, κ | Not known | Yes | Yes |
| J116 | eBioscience | Mouse IgG, κ | Not known | Weakly | Yes |
| EH12.2H7 | BioLegend | Mouse IgG1, κ | Not known | Yes | Yes |
| MIH4 | eBioscience | Mouse IgG1, κ | Not known | Yes | Yes |
| AF1086 | R&D Systems | Goat polyclonal | Mouse myeloma cell | Yes | Yes |
| | | IgG | line NS0 derived | | |
| | | | recombinant human | | |
| | | | PD1 Leu25Gln167 | | |

TABLE 3
Binding affinity of anti-PD-1Ab MIH4 for
recombinant human sPD-1 proteins

| | 2M | | | | |
|---|---|---|---|---|---|
| Ligand (on chip) | PDL-1 | kₐ, (1/Ms) | k_{d}, (1/s) | K_{D}, nM | Chi² |
| hPD1(25-167)-His | No | 1.3 × 10⁵ | 3.8 × 10⁻⁴ | 2.8 | 0.16 |
| hPD1(25-167)-His | Yes | 1.2 × 10⁵ | 3.4 × 10⁻⁴ | 2.9 | 0.08 |
| hPD1(25-167)-3S-IG | No | 4.8 × 10⁵ | 2.4 × 10⁻⁴ | 0.5 | 0.14 |

Association constant (kₐ), dissociation constant (k_{d}) and binding constant (K_{D}) for monomeric (hPD1(25-167)-His) and dimeric (hPD1(25-167)-3S-IG) sPD-1 proteins.
Chi²: goodness of fit.

In order to achieve the desired assay sensitivity and dynamic range in the presence of nivolumab, a design of experiments (DoE) study was conducted using the central composite design to determine the optimal concentrations of the capture and detection Abs. A wide range of capture and detection Ab concentrations were evaluated in this study (data not shown). With the aid of multi-variance analysis utilizing JMP 8.0 (SAS, NC, USA), the capture and detection Abs concentrations were selected at 4 and 3 μg/m1 for the assay, respectively.

### EXAMPLE 3

sPD-1 Meso Scale Discovery (MSD) Assay

Anti-PD-1 Ab MIH4 (available commercially under Catalog No. 17-9969-41 from eBiosciences, San Diego, Calif.) was labeled with biotin at 1:20 (Ab: biotin) molar ratio using an EZ-Link NHS-PEG4-Biotin kit (Thermo Fisher, Waltham, Mass.). An anti-PD-1 Ab, AF1086 from R&D Systems (available commercially under Catalog No. AF1086), was labeled with ruthenium at 1:12 (Ab:ruthenium) molar ratio, using the MSD SULFO-TAG™ NHS-Ester kit. Labeling in both cases was done following the kits' instructions.

Streptavidin plates (MSD) were incubated with BLOCKER™ Casein in PBS (Thermo Fisher Scientific, Waltham, Mass.) at room temperature (RT) for 1 h, and washed 4× with a wash buffer (1× phosphate buffered saline (PBS) with 0.05% TWEEN®-20). These plates were then coated with biotinylated anti-PD1 MIH4 (4.0 μg/ml in 1× PBS) at RT for 2 h on the day of study (fresh plate), or prepared prior to the study and treated with STABILCOAT® per manufacturers recommendations (Surmodics, Eden Prairie, Minn.). These pre-coated plates were stored at 4° C. in a sealed airtight pouch with desiccant.

During assay development and validation, each analytical run consisted of one set of standards, a blank, QCs (HQC, MQC, LQC, and LLOQ) and study samples. On the day of study, the standards, QCs and study samples were diluted 1:4 with Diluent 2 (MSD). All samples were analyzed in duplicates unless specified otherwise. 50 μl of standard, blank, QC, or study sample was incubated in a MIH4-biotin coated plate at room temperature for 2 h with shaking (about 600 rpm). The plate was washed, incubated with 50 μl of the SULFO-TAG™-labeled anti-PD-1 detection Ab, AF1086 (3.0 μg/ml in BLOCKER™ Casein), at room temperature for 1 h with shaking. Following a wash step, 150 μl of 2× Read Buffer Solution (MSD) were added into each well, and the plate was read on a MSD SECTOR® 6000 Imager.

### Data Analysis

The conversion of ECL signal units to concentrations for the study samples and QCs was performed using SoftMax Pro (version 5.4.1, Molecular Devices, Sunnyvale, Calif.). The ECL signal vs. concentration relationship was regressed according to a four-parameter logistic regression model with a weighting factor of 1/y². Goodness of curve fit was determined on the following bases: (1) significance of the parameters included in the model (95% confidence limits that do not contain “0” for the estimate of maximum response, EC₅₀ and slope); (2) p-value for the General Linear Hypothesis Test for goodness of model fit >0.001 (R² for these curves were greater than 99% so the lack of fit test was conducted at a low significance level to prevent the test from being too sensitive to small departures); (3) residual plots from the regression model that show random scatter around “0”; (4) % RE values within 20% over the range of the standards. The residual plots also provided information on the appropriate weight—the unweighted model and model with 1/y weights showed variances in the responses that increased with concentration; the weight of 1/y² provided residual plots that showed greater randomness of the residuals around “0”. Based on these criteria, the four-parameter logistic regression model with a weight of 1/y² was considered appropriate.

Variance components methods (Milliken and Johnson, 1984) in the context of analysis of variance (ANOVA) models were used for estimates of assay precision, expressed as coefficient of variation (CV) relative to the overall mean predicted concentration [% CV=100(StdDev/mean)^{1/2}]. Intra-assay (within replicates on the same plate) or inter-assay (between different plates) imprecision was defined by variance derived from back-calculated concentrations of QCs. Specifically, the estimates of inter- and intra-assay % CV were calculated as the “ANOVA Variance Components” for “Intrabatch” (s_{w}²) and “Interbatch” (s_{b}²) variance, respectively (DeSilva et al., 2003). Total variation in Table 4 is the sum of the two variance components (s_{b}² +s_{w}²).

Total error (TE) was calculated as the sum of the absolute value of percent deviation from nominal (bias) and the total variance. All analyses were performed using SAS version 9.2.

### Results

Standard Curve and Assay Range

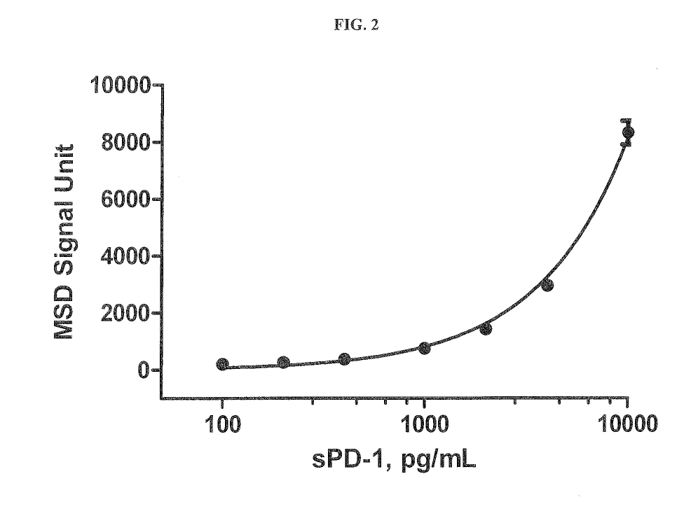

Fit-for-purpose method development and validation was conducted according to published white papers (Lee et al., 2006; Lee et al., 2009). Seven validation runs were performed over four different days to determine the analytical range of the assay. Each run consisted of one set of standards and blank, and at least two replicates of the QC and LLOQ samples. The validated analytical range of the assay was 100-10,000 pg/ml, defined by the lowest and highest non-zero points of the standard curve with acceptable precision and accuracy (FIG. 2). The LLOQ was selected close to the concentration of endogenous sPD-1 levels in normal human sera (about 100 pg/ml). Since this assay could measure sPD-1 in the presence of nivolumab (see Example 5), the levels of total sPD-1 post drug treatment might increase significantly since the clearance of analyte-Ab complex were expected to be slower (Zhang et al., 2012). Therefore, the ULOQ was selected at 10,000 pg/ml so that the calibration curve covers a wide range of concentrations. The standard curve imprecision is ≦15% in total variance; and accuracy is ≦15% in percent deviation from nominal for all back-calculated standard concentrations.

Assay Precision and Sensitivity

High, mid and low QC samples and were used to evaluate assay precision and accuracy during the validation runs. The results of these studies are summarized in Table 4. The intra- and inter-assay imprecision was ≦15%, which was defined by intra- and inter-assay variance derived from back-calculated concentrations of QCs. The assay bias or percent deviation was <10%. The accuracy of the assay, defined by percent of total error of LQC, MQC and HQC, was <20% which satisfied the targeted criteria based on internal standard operating procedures.

A LLOQ sample was also included in the same set of validation runs and was used to define the assay sensitivity to be 100 pg/ml. The intra- and inter-assay imprecision of the LLOQ was <25% with a total variance of 26.5%.

Detection of Endogenous sPD-1 Protein and Study of Matrix Effect in Normal and Cancer Sera

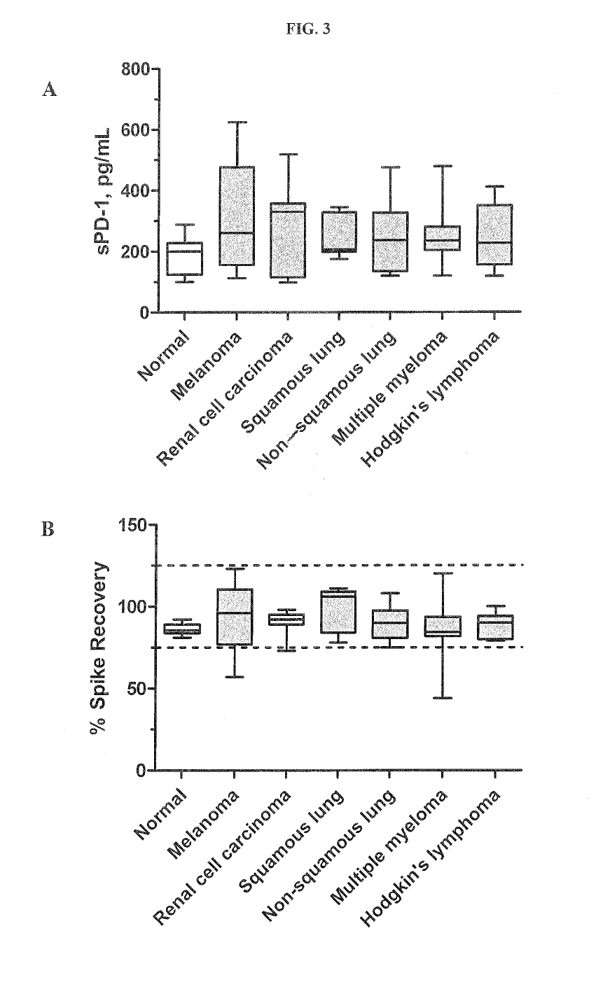

Commercial serum samples from 15 normal individuals and 58 cancer patients from 6 cancer types were surveyed. Greater than 98% of all tested samples had measurable levels of sPD-1. In this small set of samples, serum sPD-1 levels in melanoma and renal cell carcinoma patients appeared to be higher than those in normal individuals (FIG. 3A). The mean±SD values of sPD-1 levels in normal, melanoma and renal cell carcinoma patients are 188.0±16.1 (N=15), 319.2±45.6 (N=15), and 278.7±45.7 (N=11) pg/ml, respectively (p <0.05, normal vs. melanoma or renal cell carcinoma).

TABLE 4
Assay accuracy and precision of QC samples in human sera

| | LLOQ | LQC | MQC | HQC |
|---|---|---|---|---|
| Nominal concentration (ng/ml) | N/A | 500 | 1500 | 5000 |
| Mean observed concentration (ng/ml) | 101.0 | 526.8 | 1583.2 | 5482.5 |
| % Deviation | N/A | 5.3 | 5.5 | 9.7 |
| Inter-assay imprecision (% CV) | 14.8 | 5.1 | 1.6 | 5.2 |
| Intra-assay imprecision (% CV) | 22.0 | 4.2 | 2.7 | 5.4 |
| Total variation (% CV) | 26.5 | 12.9 | 6.9 | 7.5 |
| Total error (%) | N/A | 18.3 | 12.5 | 17.1 |
| n | 51 | 24 | 24 | 24 |
| Number of runs | 7 | 7 | 7 | 7 |

N/A: not applicable

The potential effect of different sample matrices were studied by spiking 500 pg/ml hPD1(25-167)-His protein into serum samples from normal and cancer individuals. No significant bias was observed in % spike recovery in these samples, although % spike recovery in cancer sera appeared to be more variable, especially in samples from multiple myeloma patients (FIG. 3B).

Dilution Linearity in Normal and Cancer Sera

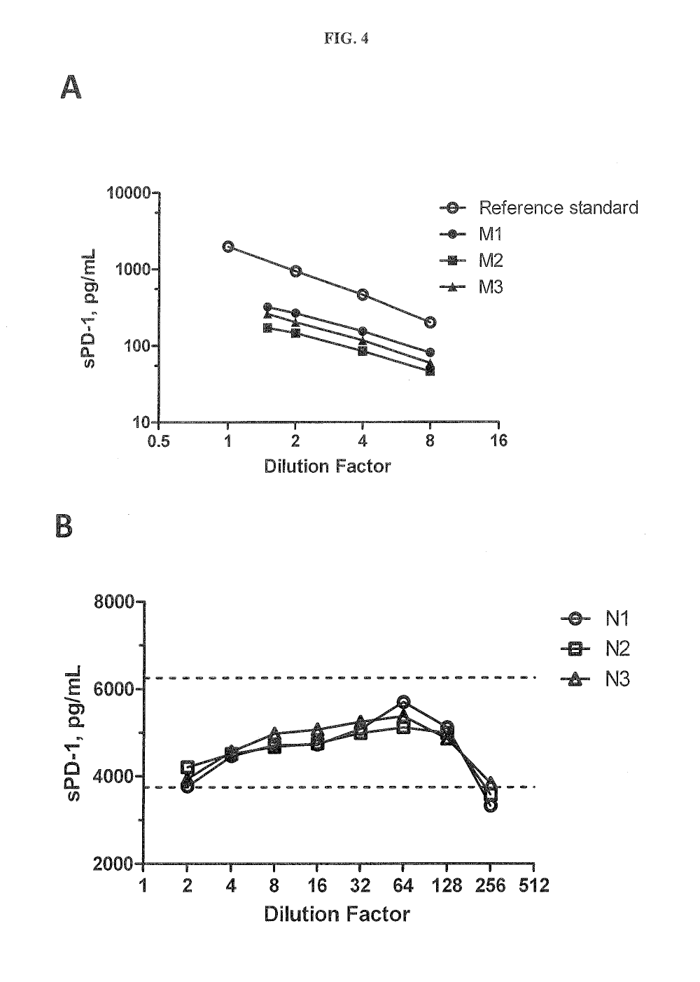

To assess sample dilution linearity, three melanoma serum samples with high levels of endogenous sPD-1 were diluted 1:1.5 to 1:8 in assay buffer. The dilution curve of these melanoma samples were parallel to that of the reference standard in SERASUB® solution and diluted in the same concentration range (FIG. 4A). Dilution linearity was also observed in two serum samples with high levels of endogenous sPD-1 from hematologic malignancy patients which were diluted 1:4 to 1:128 (data not shown). Together, these results also demonstrated that concentration-response relationship of the endogenous sPD-1 protein in sample matrix was similar to that of reference standards in assay buffer (proof of “parallelism”; Lee et al., 2009).

Dilution linearity was also demonstrated with three normal human serum samples each spiked with 5,000 pg/ml hPD1(25-167)-His protein and then serial diluted in assay buffer starting from 1:2. The back-calculated concentrations of samples diluted 1:4 to 1:128 were within 25% deviation from the nominal (FIG. 4B), suggesting no significant matrix effect in this dilution range (Lee et al., 2006; 2009). Based on results from this study, the assay's minimal required dilution factor (MRD) was determined to be 4.

### EXAMPLE 4

Optimization of sPD-1 Meso Scale Discovery (MSD) Assay

QC Sample and Endogenous Analyte Stability

To support clinical studies, the short term stability of QCs, including the LLOQ sample (a pooled human serum sample with endogenous analyte), was evaluated at different storage temperatures and through multiple freeze/thaw (F/T) cycles, and results from each of the tested conditions were compared to QC ranges established during assay validation. The effects of storage condition and F/T cycles on QC sample stability are tabulated in Table 5, with the back-calculated concentration values (pg/ml) of QC samples kept at room temperature (RT), −4° C., −20° C., or −80° C. shown in 5A, and the back-calculated concentration values of QC samples subjected to different number of F/T cycles shown in 5B. The QC range from assay validation studies is included in each table. Results are shown as mean concentration value and % CV (n=2).

As shown in Table 5, bench top stability was demonstrated at room temperature and 2-8° C. for up to 24 h, at −20° C. for up to 2 weeks, and at −80° C. for up to 4 months. Freeze/thaw cycle stability was tested by comparing data from QC samples subjected to multiple F/T cycles to those from a freshly prepared control set, and showed that both QC and test samples can withstand at least 5 F/T cycles. Together, these results informed sample handling during clinical studies.

Method Optimization and Robustness

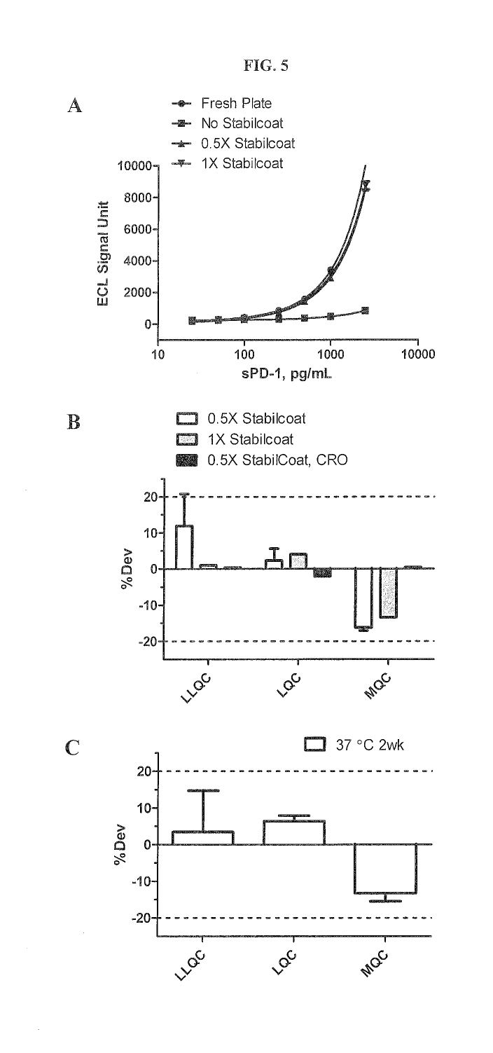

Large clinical studies often involve multiple sites and span several years. To ensure consistency of assay performance overtime and to reduce assay duration, it was investigated whether assay plates can be coated with capture Abs prior to assay day. The analytical performance of the streptavidin plates (MSD) pre-coated with capture Ab were evaluated by comparing standard and QC sample performance using pre-coated plates stored at 4° C. or 37° C. overnight with those using freshly-coated streptavidin plates. Results from these studies demonstrated that assay standard curve and QC performance of pre-coated plates and freshly coated plates were comparable as long as the pre-coated plates were treated with STABILCOAT® (FIGS. 5A and B).

TABLE 5
Storage condition and freeze/thaw effects on QC sample stability
A.

| | Validation Range, pg/ml | | 24 h at RT | | 24 h at 4° C. | | 2 weeks at −20° C. | | 4 months at −80° C. | |
|---|---|---|---|---|---|---|---|---|---|---|
| | Low value | High value | pg/ml | % CV | pg/ml | % CV | pg/ml | % CV | pg/ml | % CV |
| LLOQ | 49 | 153 | NT | | NT | | 86 | 9.4 | NT | |
| LQC | 412 | 600 | 447 | 2 | 482 | 3.4 | 504 | 3.3 | 474 | 1.3 |
| MQC | 1142 | 1784 | 1483 | 1.4 | 1478 | 1.1 | 1513 | 3.9 | 1464 | 0.7 |
| HQC | 4148 | 6150 | 5224 | 1.3 | 5198 | 2.9 | 5154 | 7.1 | 5180 | 1.8 |

B.

| | Validation Range, pg/ml | | 0 FT | | 1X FT | | 2X FT | | 3X FT | | 4X FT | | 5X FT | |
|---|---|---|---|---|---|---|---|---|---|---|---|---|---|---|
| | Low value | High value | pg/ml | % CV | pg/ml | % CV | pg/ml | % C V | pg/ml | % CV | pg/ml | % CV | pg/ml | % CV |
| LLOQ | 49 | 153 | 84 | 16.1 | NT | | 60 | 11.7 | 95 | 5.6 | 98 | 12.2 | 92 | 21.7 |
| LQC | 412 | 600 | 505 | 0.7 | 469 | 1.1 | 503 | 5.7 | 539 | 1 | 538 | 1 | 546 | 2.8 |
| MQC | 1142 | 1784 | 1532 | 1 | 1358 | 1.4 | 1584 | 0.6 | 1507 | 1.8 | 1567 | 0.1 | 1606 | 0.5 |
| HQC | 4148 | 6150 | 5067 | 0.1 | 4954 | 1.2 | 5328 | 9.3 | 5435 | 7.3 | 5380 | 2.9 | 5310 | 6.9 |

NT: not tested.

The stability of these pre-coated plates with STABILCOAT® was evaluated by an accelerated stability assessment approach (Anderson et al., 1991; Weiss et al., 2009) and real time assessment. During the accelerated stability assessment, the pre-coated plates with STABILCOAT® were kept in an air-tight pouch with desiccants for 1 or 2 weeks at 37° C., and then the QC sample performance was evaluated. The results showed that the pre-coated plates with STABILCOAT® are stable for up to 2 weeks at 37° C. Based on the Arrhenius equation (Anderson et al., 1991; Weiss et al., 2009), the estimated stability of these pates stored at 2-8° C. is about 1 year (FIG. 5C). In these studies, percent deviation (% Dev) of each QC was calculated against its mean value from validation studies using fresh plates.

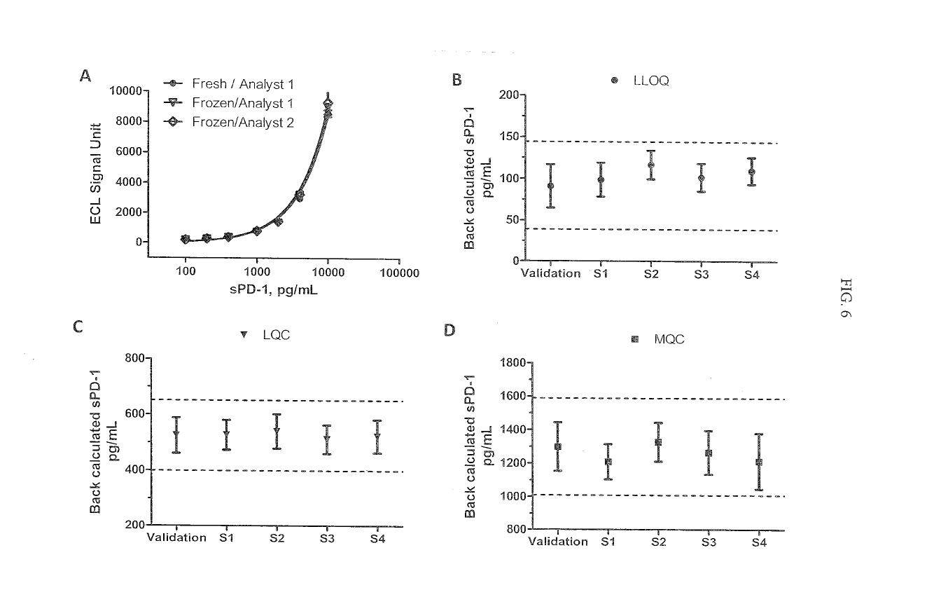

The use of bulk-prepared frozen reference standard samples can further improve the consistency of assay performance and reduce assay duration. The standard curves generated using bulk-prepared frozen standards were compared with those using freshly made standards. The study results showed that the frozen standards can be kept in aliquots at −80° C. for up to 2 months without any significant changes in quality (FIG. 6A). In addition, FIG. 6A also showed consistent assay performance among analytical runs conducted by two different analysts on different days. Together, these results demonstrate that the sPD-1 assay described herein is very robust.

Further demonstration of assay robustness came from sample analyses of 56 analytical runs from 4 clinical studies. QC sample data from all 56 runs were within the acceptance criteria established during assay validation. These analytical runs were performed by 3 analysts and using 2 reference standard lots and 3 labeled coating and detection Ab lots (FIGS. 6B-D).

### EXAMPLE 5

Effects of PDL-1, PDL-2 and Nivolumab on the sPD-1 MSD Assay

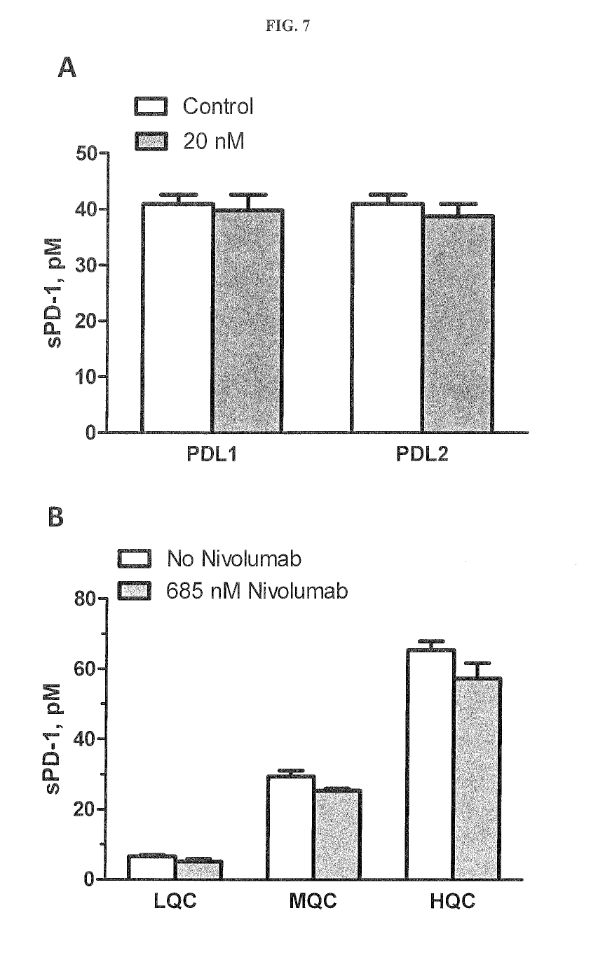

To assess whether the endogenous ligands of PD-1 protein interfere with the assay, sPD-1 protein was measured in a melanoma serum sample in the absence or presence of varying concentrations of human PDL-1 and PDL-2 proteins. Soluble PDL-1 in human sera has been reported in the low ng/ml range (Chen et al., 2011); levels of soluble PDL-2 in sera are currently not known. As shown in FIG. 7A, PDL-1 or PDL-2, up to 1000 ng/ml, did not have a significant effect on sPD-1 measurement. This study was repeated with three additional melanoma serum samples, and similar results were observed.

In a separate study, the effect of nivolumab (1-100 μg/ml ) on the assay was evaluated. As shown in FIG. 7B, nivolumab at 100 μg/ml had minimal interference on the assay performance (about 15-20% reduction in back-calculated QC values). Together, these results demonstrated that the sPD-1 assay is a “total assay,” which is capable of measuring circulating human sPD-1 regardless of the presence of concurrently bound nivolumab, PDL-1 or PDL-2.

### REFERENCES

Agata Y, Kawasaki A, Nishimura H, Ishida Y, Tsubat T, Yagita H, et al. (1996) Expression of the PD-1 antigen on the surface of stimulated mouse T and B lymphocytes. Intl Immunol 8(5):765-72.
Anderson G, Scott M. (1991) Determination of product shelf life and activation energy for five drugs of abuse. Clin Chem 37(3):398-402.
Chen Y, Wang Q, Shi B, Xu P, Hu Z, Bai L, et al. (2011) Development of a sandwich ELISA for evaluating soluble PD-L1 (CD274) in human sera of different ages as well as supernatants of PD-L1+cell lines. Cytokine 56(2):231-8.
Cheng H Y, Kang P J, Chuang Y H, Wang Y H, Jan M C, Wu C F, et al. (2014) Circulating programmed death-1 as a marker for sustained high hepatitis B viral load and risk of hepatocellular carcinoma. PLoS One 9(11):e95870.
Cheng X, Veverka V, Radhakrishnan A, Waters L C, Muskett FW, Morgan S, et al. (2013) Structure and interactions of the human programmed cell death 1 receptor. J Biol Chem 288(17):11771-85.
DeSilva B, Smith W, Weiner R, Kelley M, Smolec J, Lee B, et al. (2003) Recommendations for the bioanalytical method validation of ligand-binding assays to support pharmacokinetic assessments of macromolecules. Pharm Res 20(11):1885-900.
Dong H, Strome S E, Salomao D R, Tamura H, Hirano F, Flies D B, et al. (2002) Tumor-associated B7-H1 promotes T-cell apoptosis: A potential mechanism of immune evasion. Nat Med 8(8):793-800.
Dublin C S (2012) Cautious optimism surrounds early clinical data for PD-1 blocker. Nature Biotech 30(8):729-30.
Engvall E (1980). [28] Enzyme immunoassay ELISA and EMIT. Methods Enzymol 70:419-39.
Forster R J, Bertoncello P, Keyes T E 92009) Electrogenerated chemiluminescence. Annu Rev Anal Chem 2:359-85.
Freeman G J, Long A J, Iwai Y, Bourque K, Chernova T, Nishimura H, et al. (2000) Engagement of the PD-1 immunoinhibitory receptor by a novel B7 family member leads to negative regulation of lymphocyte activation. J Exp Med 192(7):1027-34.
Greisen S, Rasmussen T, Stengaard-Pedersen K, Hetland M, Horslev-Petersen K, Hvid M, et al. (2014) Increased soluble programmed death-1 (sPD-1) is associated with disease activity and radiographic progression in early rheumatoid arthritis. Scand J Rheumatol 43(2):101-8.
Hamid O, Carvajal R D (2013) Anti-programmed death-1 and anti-programmed death-ligand 1 antibodies in cancer therapy. Expert Opin Biol Ther 13(6):847-61.
Hamid O, Robert C, Daud A, Hodi F S, Hwu W J, Kefford R, Wolchok J D, Hersey P (2013) Safety and tumor responses with lambrolizumab (anti-PD-1) in melanoma. N Engl Med 369(2):134-44.
Haulenbeek J, Piccoli S P (2014) Conjugated critical reagent characterization for ligand-binding assays: using MALDI-TOF-MS as an orthogonal tool to assess assay performance. Bioanalysis 6(7):983-92.
Lee J W (2009) Method validation and application of protein biomarkers: basic similarities and differences from biotherapeutics. Bioanalysis (8):1461-74.
Lee J W, Devanarayan V, Barrett Y C, Weiner R, Allinson J, Fountain S, et al. (2006) Fit-for-purpose method development and validation for successful biomarker measurement. Pharm Res 23(2):312-28.
McDermott D F, Atkins M B (2013) PD-1 as a potential target in cancer therapy. Cancer Med 2(5):662-73.
McDermott D F, Jimeno A (2015) Pembrolizumab: PD-1 inhibition as a therapeutic strategy in cancer. Drugs Today (Barc) 51(1):7-20.
Milliken G A, Johnson D E (1984) Analysis of Messy Data. CA, USA: Wadsworth, Inc.
Nielsen C, Ohm-Laursen L, Barington T, Husby S, Lillevang S T (2005) Alternative splice variants of the human PD-1 gene. Cell Immunol 235(2):109-16.
Nishimura H, Agata Y, Kawasaki A, Sato M, Imamura S, Minato N, et al. (1996) Developmentally regulated expression of the PD-1 protein on the surface of double-negative (CD4-CD8-) thymocytes. Int Immunol 8(5):773-80.
Pardoll D, Drake C (2012) Immunotherapy earns its spot in the ranks of cancer therapy. J Exp Med 209(2):201-9.
Shinohara T, Taniwaki M, Ishida Y, Kawaichi M, Honjo T (1994) Structure and Chromosomal Localization of the Human PD-1 Gene (PDCD1). Genomics 23(3):704-6.
Topalian S L, Drake C G, Pardoll D M (2012) Targeting the PD-1/B7-H1(PD-L1) pathway to activate anti-tumor immunity. Curr Opin Immunol 24(2):207-12.
Voller A, Bartlett A, Bidwell D E (1978) Enzyme immunoassays with special reference to ELISA techniques. J Clin Pathol 31(6):507-20.
Wan B, Nie H, Liu A, Feng G, He D, Xu R, et al. (2006) Aberrant regulation of synovial T cell activation by soluble costimulatory molecules in rheumatoid arthritis. J Immunol 177(12):8844-50.
Wang C, Thudium K B, Han M, Wang X-T, Huang H, Feingersh D, et al. (2014) In vitro characterization of the anti-PD-1 antibody nivolumab, BMS-936558, and in vivo toxicology in non-human primates. Cancer Immunol Res 2(9):846-56.
Weiss W F, Young T M, Roberts C J (2009) Principles, approaches, and challenges for predicting protein aggregation rates and shelf life. J Pharm Sci 98(4):1246-77.
Zhang L, McCabe T, Condra J H, Ni Y G, Peterson L B, Wang W, et al. (2012) An anti-PCSK9 antibody reduces LDL-cholesterol on top of a statin and suppresses hepatocyte SREBP-regulated genes. Int J Biol Sci 8(3):310-27.
Zhang X, Schwartz J-C D, Guo X, Bhatia S, Cao E, Chen L, et al. (2004) Structural and functional analysis of the costimulatory receptor Programmed Death-1. Immunity 20(3):337-47.

What is claimed is:

1. An immunoassay for quantifying total soluble Programmed Death-1 (sPD-1) in a sample solution comprising:
(a) immobilizing an anti-PD-1 capture antibody in multiple discrete locations on a solid support, wherein each location contains electrodes designed for applying a voltage waveform effective to trigger electrochemiluminescence (ECL) in a suitable electrochemiluminescent substrate, and further wherein:
(i) the capture antibody binds specifically to sPD-1 in both monomeric and dimeric forms; and
(ii) binding of the capture antibody to PD-1 is essentially the same in the presence or absence of a therapeutic anti-PD-1 antibody, PDL-1 and/or PDL-2;
(b) contacting the immobilized capture antibody in a sample location with an aliquot of the sample solution and in reference locations with aliquots of a series of reference solutions containing known quantities of sPD-1 in monomeric form, under conditions to allow the immobilized capture antibody to bind to sPD-1 in the sample and reference solutions;
(c) contacting each sample and reference location with an electrochemiluminescent-labeled anti-PD-1 detection antibody under conditions to allow the detection antibody to bind to sPD-1 bound by the capture antibody in the sample and reference locations, wherein
(i) the detection antibody binds specifically to sPD-1 in both monomeric and dimeric forms;
(ii) the detection antibody binds to a different epitope on sPD-1 than the epitope bound by the capture antibody; and
(iii) binding of the detection antibody to PD-1 is essentially the same in the presence or absence of a therapeutic anti-PD-1 antibody, PDL-1 and/or PDL-2;
(d) determining the relative quantities of the detection antibody bound to the sample location and the reference locations by measuring ECL; and
(e) quantifying the amount of sPD-1 in the sample solution by comparing the ECL from the detection antibody bound to the sample location relative to the ECL from the detection antibody bound to the reference locations.
